SEQUENCE LISTING (1 sequence)

SEQ ID NO: 1 - protein, 29 aa, Homo sapiens
  PEPTIDE (1)...(29): glucagon mature peptide
HSQGTFTSDYSKYLDSRRAQDFVQWLMNT

(19) United States
(12) Patent Application Publication
(10) Pub. No.: US 2014/0378381 A1
(43) Pub. Date: Dec. 25, 2014

(54) STABILIZED GLUCAGON NANOEMULSIONS

(71) Applicant: LATITUDE PHARMACEUTICALS, INC., San Diego, CA (US)

(72) Inventors: Andrew Xian Chen, San Diego, CA (US); Norman Keith Orida, San Diego, CA (US); Hailiang Chen, San Diego, CA (US); Hau Huu Dang, San Diego, CA (US)

(21) Appl. No.: 14/315,212

(22) Filed: Jun. 25, 2014

Related U.S. Application Data
(63) Continuation-in-part of application No. PCT/US2012/071326, filed on Dec. 21, 2012.
(60) Provisional application No. 61/581,610, filed on Dec. 29, 2011.

(51) Int. Cl. A61K 9/107; A61K 47/26; A61K 47/18; A61K 47/20; A61K 38/26; A61K 47/24

(52) U.S. Cl. CPC A61K 9/107; A61K 38/26; A61K 47/24; A61K 47/183; A61K 47/20; A61K 47/26
USPC 514/11.7

(57) ABSTRACT

The present invention provides an oil-in-water nanoemulsion containing glucagon, an oily phase, and an aqueous phase, wherein the glucagon is physically and chemically stable and the nanoemulsion is suitable for administration by manual injection or by a pump to treat hypoglycemia.

### CROSS-REFERENCES TO RELATED APPLICATIONS

The present application is a Continuation-in-Part of PCT/US2012/071326, filed Dec. 21, 2012, which application claims priority to U.S. Provisional Patent Application No. 61/581,610, filed Dec. 29, 2011, the contents of both applications are hereby incorporated by reference in their entireties for all purposes.

### FIELD OF THE INVENTION

This disclosure relates to stabilized glucagon nanoemulsions.

### BACKGROUND OF THE INVENTION

Glucagon, a hormone secreted by the pancreas, is a polypeptide consisting of a single chain of 29 amino acids and has a molecular weight of 3,485 Da. Both synthetic and recombinant glucagon are available with suitable purity that can enable their use as pharmaceuticals. Glucagon is not absorbed orally and is therefore administered by injection.

Medically, glucagon is used to treat hypoglycemia (characterized by lower than normal blood glucose concentrations). Hypoglycemia is common in Type-1 diabetic patients and insulin users. Mild hypoglycemia causes anxiety, sweating, tremors, palpitations, nausea, and pallor. In severe hypoglycemia, the brain is starved of the glucose it needs for energy, leading to seizures, coma or even death. Severe hypoglycemia is a life-threatening emergency that requires immediate medical intervention, for which the current standard of care is glucagon injection.

Upon injection, glucagon stimulates the liver to convert stored glycogen into glucose, which is released into the blood. The onset of action for glucagon occurs 5-20 minutes after injection. The half-life of glucagon in blood is 3 to 6 minutes, which is similar to insulin.

Glucagon has an isoelectric point of 7.1 and is thus insoluble in water at physiological pH (pH 4-8) and precipitates in pH-neutral aqueous solutions. In aqueous solutions of pH 3 or less, it is initially soluble, but will aggregate to form a gel within an hour. The gelled glucagon consists predominantly of β-sheet fibrils that are induced by the hydrophobicity and the inter- and intra-chain hydrogen bond forming potential of the peptide (Chou, P. Y. et al. 1975. Biochemistry 14(11):2536-2541). The aggregated glucagon is not suitable for injection because the gel can clog a hypodermic needle and, if intravenously administered, blood vessels. To slow the aggregation process, an acidic (pH 2-4) formulation is commonly used to maintain glucagon in a relatively aggregation-free state for a short time. Such acidic formulations must be injected immediately after preparation as the glucagon will aggregate (Product Insert for GlucaGen® Hypokit for injection [glucagon [rDNA origin]).

In addition to its physical instability, glucagon undergoes various types of chemical degradation. In aqueous solution, it rapidly degrades to form several degradation products. At least 16 degradation products of glucagon have been reported with the major degradation pathways being aspartic acid cleavage at positions 9, 15, and 21 and glutaminyl deamidation at positions 3, 20 and 24 (Kirsch, L. E., et al. 2000. International Journal of Pharmaceutics, 203:115-125). The chemical degradation of glucagon is rapid and complex. For example, in an acidic solution (pH 2-4) required to dissolve glucagon and prevent its aggregation, about 5-70% of the glucagon decomposes into numerous degradation products within 24 hours at 37° C. (U.S. Patent Application Publication No. 2011/0097386). Preventing glucagon degradation in an aqueous environment is very difficult and no effective method has yet been developed to slow the aspartic cleavage and glutaminyl deamidation of glucagon in an aqueous environment. This instability has limited the medical utility of the currently available glucagon formulations.

Glucagon is indicated for the treatment of severe hypoglycemia. In order to circumvent glucagon's chemical instability, the currently available glucagon drug products (e.g., GlucaGen Hypokit (glucagon hydrochloride) from Novo Nordisk and Glucagon for Injection (rDNA origin) from Eli Lilly and Company) are lyophilized and provided as two-part kits. One part is a vial containing 1 mg (1 unit) of glucagon and 49 mg of lactose in a dry lyophilized solid mass (“cake”) and the other part is a syringe containing a diluent comprising 12 mg/mL glycerin, water and hydrochloric acid. Lyophilization provides an anhydrous environment that keeps glucagon stable by preventing aspartic acid cleavage, glutaminyl deamidation and any water-dependent degradative pathways. To use the glucagon kit, the diluent is first injected from the syringe into the cake-containing vial, which is then gently swirled to dissolve the glucagon. The reconstituted glucagon solution is then drawn back into the same syringe, which is now ready for injection. The pH of this solution is approximately 2.0-3.5. The reconstituted glucagon solution is unstable and the manufacturers recommend it to be used immediately after reconstitution and to discard any unused portion. Thus, each glucagon kit is intended only for a single and immediate use.

The proper use of the two-part glucagon kit requires a complicated multiple-step procedure that includes taking stock of the kit components, removing the cap seal, injecting the diluent into the vial, reconstituting the glucagon cake, withdrawing the glucagon solution, and administering the reconstituted solution. This cumbersome procedure could be difficult even for a normal person to perform. For someone incapacitated by hypoglycemia, the task may be extremely difficult or impossible. A delay in administering timely glucagon rescue therapy could result in death. Sadly, 6-10% of deaths of individuals with Type 1 diabetes are a result of hypoglycemia (Cryer, P. E. 2008. Diabetes 57(12): 3169-3176). Thus, a stable and ready-to-inject liquid glucagon formulation would be highly desirable for emergency hypoglycemia rescue and has the potential to save lives.

Insulin pumps have been widely used by insulin dependent diabetics for over a decade. These pumps provide a continuous flow of insulin to patients. After a meal, the user can manually increase the insulin flow to temporarily cover the post-prandial blood glucose surge, and then dial back to a slow basal maintenance flow. These pumps can be attached directly to the abdominal surface and deliver insulin directly to subcutaneously inserted small needles (e.g., the Omnipod from Insulet Corp.) or can be worn externally in close proximity to the body and deliver insulin via fine tubing through subcutaneously implanted needles (e.g., OneTouch® Ping (Animas Corp.), Revel™ (Medtronic, Inc.), and others). The subcutaneous needles may remain in place for up to a week.

The environmental conditions constraining pump use require that insulin and any other liquids that are delivered by such pumps must be stable for at least 3-7 days at body or near body temperature (30-37° C.). Newer so-called artificial pancreas devices have been developed that incorporate sensors having the ability to continuously read the patient glucose levels (so-called continuous glucose monitoring or “CGM”) and use that information to adjust insulin pump output to the requisite levels in real time. However, when too much insulin has been pump-delivered, the current versions of the insulin-only artificial pancreas do not have an effective means to rapidly counteract the drop in blood glucose and impending hypoglycemia from the already-administered insulin. In the normal individual, the body naturally counteracts rapid blood glucose decreases by releasing glucagon, but in a Type-1 diabetic patient, such function is impaired due to the diminished alpha cell activity.

A bi-hormonal closed loop pump or a true artificial pancreas is a CGM-linked insulin pump, which is capable of delivering both insulin and glucagon to the patient. When blood glucose reaches or is anticipated to reach hypoglycemic levels, the bi-hormonal pump delivers glucagon to counteract hypoglycemia. This capability allows the patient's blood glucose to be highly regulated to within euglycemic levels, as performed by the pancreas of a non-Type-1 diabetic individual.

A true bi-hormonal pump requires a liquid glucagon formulation that is stable for at least three to seven days at body or near body temperature. Furthermore, the formulation must not be irritating and cause discomfort and pain where the delivery needles are implanted. Therefore, a glucagon formulation for the artificial pancreas must not contain components which are known to be irritating or hemolytic such as low pH or lysolecithin. The formulations for the currently available two-part glucagon kits as well as a number of other emerging glucagon formulations do not meet these criteria.

Glucagon injection is also indicated for inhibiting gastrointestinal motility during certain radiological imaging procedures. The glucagon dose for this application is less than the 1 mg that comes in the two-part emergency kits. Since the two-part glucagon kit can be used only once and the un-used portion discarded, significant waste occurs any time a two-part rescue kit is used for an imaging procedure. Therefore, a new formulation that is stable in the liquid form and capable of being provided in a multiple-dose vial is very desirable. Such a multi-dose-capable, liquid formulation must contain an antimicrobial preservative to prevent microbial growth.

In summary, there is a great need for a new composition for glucagon that is:
1. Liquid and injection-ready to allow for quick administration for rescue of patients in severe hypoglycemia.
2. Stable at body or near body temperature (30-37° C.) for 3-7 days to enable practical use in a bi-hormonal insulin/glucagon pump.
3. Stable at a normal storage temperature (5 or 25° C.) for 1-2 years, a shelf-life requirement for commercial drug products.
4. Capable of providing multiple doses from the same vial to reduce waste for low dose indications such as in gastrointestinal radiological examinations.

There is no known glucagon composition in the prior art that is capable of meeting all four of the above requirements. Most stabilized glucagon formulations have been developed to prevent glucagon aggregation or gelation, i.e., to address glucagon's physical instability in the solution state. While some approaches appear to have successfully reduced glucagon aggregation, little attempt has been made to address glucagon's chemical degradation. Without reducing chemical degradation to an acceptable level, any glucagon composition will have limited application as a drug product.

To prevent glucagon aggregation, gelation or precipitation, most known formulations employ water-soluble surfactants, detergents or well-known drug solubilizers that dissolve glucagon to form a clear solution. These attempts have included: using up to a six-fold molar excess of cationic or anionic monovalent detergent (Great Britain Patent 1202607); hen egg lysolecithin (Schneider, A. B. and Edelhoch, H. J. 1972. Biol. Chem. 247: 4986-4991); lysolecithin (Robinson, R. M., et al. 1982. Biopolymers 21: 1217-1228); micelles of anionic detergent sodium dodecyl sulfate (SDS) at low pH (Wu, C.-S. C. and Yang, J. T. 1978. Biochemistry 19: 2117-2122) and SDS micelles at neutral pH (Brown, L. R. and Wuthrich, K. 1980. Biochim. Biophys. Acta 603: 298-312); cyclodextrins (Matilainen, L., et al. 2008. J. Pharm Sci. 97(7):2720-9 and Matilainen, L. et al. 2009. Eur. J. Pharm Sci. 36(4-5):412-20); lysophospholipids (1-acyl-sn-glycero-3-phosphoate ester of ethanolamine, choline, serine or threonine) or other detergents such as cetyl trimethylammonium bromide (CTAB) and SDS, etc. (European Patent 1061947); and lysophospholipid-sucrose combinations (US Patent Application 2011/0097386).

Previous efforts in addressing the glucagon aggregation problem have been based on the use of certain water-soluble surfactants or detergents. One popular example of a water-soluble surfactant used is lysophospholipid or lysolecithin. The prefix “lyso” is used for various phospholipids to indicate the absence of one of the two fatty acids in either the 1- or 2-position (FIG. 2). Lecithin is a mixture of naturally occurring phospholipids and, similarly, lysolecithin is a mixture of lysophospholipids from a natural source.

Unlike the two fatty acid-containing (i.e., diacyl) phospholipids or lecithins, which are generally insoluble in water, oily, and lacking detergent-like properties, single fatty acid (i.e., monoacyl) lysophospholipids are water-soluble and capable to dissolving oily substances because of their detergent properties. CTAB and SDS are highly water-soluble surfactants containing a single long carbon chain (like the monoacyl phospholipids) that are commonly used in household detergents.

Despite the structural variety of water-soluble surfactants used in known formulations, all solubilize glucagon by same mechanism. They reduce hydrophobic interaction between water and glucagon molecules, disrupt aggregated or β-sheet glucagon, and incorporate glucagon into micelles formed by the surfactants. The final composition of such a surfactant-solubilized glucagon is indeed a solution. Glucagon solution compositions were thought to be a desirable goal of the prior art since it was believed to be suitable for the glucagon rescue use or in a pump, whereas other liquid composition such as emulsions were regarded as unsuitable. For example, Steiner, S., et al. (U.S. Patent Application Publication No. 2011/0097386) noted that emulsions were not preferred formulations for glucagon for pump use because of their presumed inherently high viscosity.

Problematically, water-soluble surfactants are generally too toxic or irritating for use in injectable drugs. For example, lysophospholipids or lysolecithins have long been known to lyse red blood cells because of their hemolytic properties (Wilbur, K. M., et al. 1943. Journal of Cellular and Comparative Physiology 22(3):233-249). A bolus subcutaneous injection of lysophospholipids or lysolecithins will thus cause local tissue damage and great pain at the injection site. A prolonged and continued subcutaneous infusion of a lysolecithin-based glucagon, as might occur during use of a bi-hormonal insulin/glucagon pump, exacerbates the pain and irritation at the needle insertion site. Furthermore, no lysophospholipid, lysolecithin, SDS or CTAB has yet been approved by the FDA for use in subcutaneously injectable drugs (i.e., none are listed in the FDA's Inactive Ingredient List) at the time of the present invention. Moreover, although the water-soluble, surfactant-based prior art compositions have effectively reduced glucagon aggregation, none of these approaches has been shown to slow glucagon's chemical degradation in solution. In fact, water-soluble surfactants have the potential to accelerate glucagon degradation because monomeric or de-aggregated glucagon is more prone to attack by ions or water molecules in an aqueous solution than the aggregated or β-sheet form.

Kornfelt, et al. (U.S. Pat. No. 5,652,216) describe acidic pharmaceutical preparations (e.g., pH 2.8) comprising glucagon and a stabilizing amount of a pharmaceutically acceptable ampholyte, such as an amino acid or a dipeptide or a mixture thereof. The stabilizing amount was defined in the range of 0.1-50 micromolar, which is equal to 14.9 ng/mL-7.45 μg/mL for the amino acid methionine (MW=149). While Kornfelt, et al.'s invention addressed both the physical and chemical stability of glucagon, an acidic composition, can be very irritating when injected subcutaneously and is undesirable for pump use as the patients will suffer continuous and prolonged pain at the subcutaneous needle site. Kornfelt, et al. (ibid.) did not disclose any use of an emulsion composition or ampholyte in an emulsion composition for glucagon.

Therefore, there still exists a need for a liquid composition that can physically and chemically stabilize glucagon without the use of any water-soluble surfactant, acidic, toxic or irritating agent. The surprising characteristics of the present invention address this and other needs.

### SUMMARY OF THE INVENTION

In a first aspect, the present invention provides an oil-in-water nanoemulsion composition comprising glucagon, an oily phase, and an aqueous phase, wherein the glucagon is not aggregated and retains no less than 75% of its concentration after 3-7 days storage at 37° C., and wherein the oily phase is in the form of oil droplets having a mean diameter of less than about 200 nm.

In a second aspect, the invention provides a lyophilized dry composition containing glucagon, a phospholipid, a medium chain oil, and a sugar, wherein, upon mixing with water, the lyophilized dry composition forms a nanoemulsion of the invention.

In a third aspect, the invention provides a process for preparing a nanoemulsion of the invention. The process includes: combining glucagon and an aqueous phase; adding phospholipid and oil; mixing and homogenizing to form a nanoemulsion having an average droplet size of no more than 200 nm in diameter; and passing the nanoemulsion through a 0.2-micron filter. Methods for preparing the dry compositions of the invention further include lyophilizing the nanoemulsion.

In a fourth aspect, the invention provides a method of treating a patient in need of glucagon. The method includes administering a composition of the invention to the patient.

This invention relates to a surprising discovery that enables glucagon to be solubilized and maintained in an aggregation-free state by oil droplets of an oil-in-water nanoemulsion, wherein the oil droplets have a Total Droplet Surface Area exceeding a certain minimum, or “Critical Droplet Surface Area.”

The invention relates to the surprising finding that glucagon is chemically stabilized by the oil droplets of an oil-in-water nanoemulsion wherein more than 50% of the glucagon is associated with the oil droplets and the total dissolved ion concentration in such nanoemulsion is below certain limit, or “Critical Ion Content Limit.”

In one embodiment, the nanoemulsion does not have any added organic solvent, lysolecithin, lysophospholipid, water-soluble surfactant or cyclodextrin and yet is able to solubilize glucagon and maintain it in an aggregation-free state. Since the oil droplets are insoluble in water and completely lack surfactant or detergent properties, the solubilization or de-aggregation mechanism provided by the nanoemulsion composition is different from any other compositions disclosed in the prior art which rely on an solvent, lysolecithin, lysophospholipid, water-soluble surfactants or cyclodextrins to solubilize/de-aggregate glucagon. The nanoemulsion composition is therefore unexpected and could not have been predicted based on previously known systems and methods.

### BRIEF DESCRIPTION OF THE DRAWINGS

FIG. 1 is a schematic structure of glucagon.

FIG. 2 illustrates the structures of phospholipids and lysophospholipids. R1 or R2 denotes the alkyl chain of a fatty acid and X is choline, glycerol, ethanolamine or serine, as respectively, in phosphatidylcholine (PC), phosphatidylglycerol (PG), phosphatidyletanolamine (PE), or phosphatidylserine (PS). Lecithin is a mixture of PC with other phospholipids. PC or a lecithin containing no less than 75% PC by weight PC is preferred phospholipid for the nanoemulsion of this invention. Also shown below are examples of phosphatidylcholine (dimyristoyl-phosphatidylcholine or DMPC) and a lysophosphatidylcholine (lysomyristoyl-phosphatidylcholine or LMPC), which is the preferred solubilizer of US Patent Application 2011/0097386.

FIG. 3 shows representative chromatogram of a freshly prepared (Panel 1) and a degraded glucagon sample (Panel 2). This analytical method is capable of separating and quantitating the major degradation products of glucagon, i.e., the numerous aspartic cleavage and glutaminyl deamidation degradation products and oxidation products (Panel 2). FIG. 3. Panel 3 compares the degree of degradation products present in the nanoemulsion of the present invention (F-22) with the Glucagon for Injection (rDNA origin) from Eli Lilly & Company after 2 months storage at 25° C.

FIG. 4. Rate profiles for glucagon degradation in a nanoemulsion of the present invention at 40° C. across various pH values.

FIG. 5. Photograph depicting the semi-transparent appearance of a glucagon nanoemulsion of the present invention (F-22).

FIG. 6 shows the appearance of a lyophilized glucagon nanoemulsion (F-22) and the semi-transparent appearance of a reconstituted liquid nanoemulsion (after adding water to said lyophilized nanoemulsion).

FIG. 7 shows the pharmacodynamic profile in mice of a glucagon nanoemulsion (F-22) compared to that of Glucagon for Injection (rDNA origin) from Eli Lilly & Company (see Example 15).

### DETAILED DESCRIPTION OF THE INVENTION

### I. General

The present invention relates to liquid, ready-to-inject compositions in which glucagon is both physically and chemically stable. Specifically, the present invention discloses an oil-in-water nanoemulsion composition containing, consisting essentially of, or consisting of glucagon that is
(a) a liquid that is substantially free of gelled or precipitated glucagon,
(b) pH neutral or slightly acidic,
(c) of low viscosity and readily injectable through a fine needle,
(d) free of any water-soluble surfactant including lysophospholipid or lysolecithin,
(e) physically and chemically stable for at least 3-7 days at 30-37° C.,
(f) physically and chemically stable for at least 1 year at a normal storage temperature (e.g., 5 or 25° C.), and
(g) capable of providing multiple doses from the same vial.

### II. Definitions

As used herein, the various terms shall have the following definitions:

As used herein, “about” describes a quantity with a range covering 10% expansion from both sides of the target value. For example, “about 100” means any value between 90 and 110 including 90 and 110.

The phrase “Acceptable Injectability Criterion” as used herein is a quantitative definition of the injectability of a liquid formulation from a particular syringe/needle configuration. In the present invention, the Acceptable Injectability Criterion is a force of 1.5 pounds to extrude the particular liquid at about 0.9 cc/min from a 3 mm I.D. insulin syringe equipped with a 28 G ½ inch needle (e.g., U-100 3/10 cc Insulin Syringe (Becton, Dickinson and Co.); 28 G ½ needle (Becton, Dickinson and Co.)). This Criterion represents a normal force that can be applied comfortably by a human hand to a syringe or delivered by a medical device pump and resulting extrusion rate.

As used herein, “aggregated glucagon” or “glucagon aggregation” means presence of visible glucagon particles, fibrils or gelation in a liquid composition containing glucagon. Glucagon aggregates, fibrils or gel cannot pass through a 0.2-micron filter membrane.

As used herein, “antimicrobial preservative” is a pharmaceutical additive that can be added to a liquid composition to inhibit the growth of bacteria and fungi. The antimicrobial preservatives useful in the present invention include, but are not limited to, cresols, phenol, benzyl alcohol, ethanol, chlorobutanol, parabens, imidura, benzalkonium chloride, EDTA or its salt or a combination thereof.

An “antioxidant” is a pharmaceutical additive that can be added to a liquid composition to prevent oxidation of the active drug or an inactive component. Antioxidants include reducing agents, metal ion chelating agents and inert gases.

The term “aqueous phase” refers to a water solution containing the water-soluble additives such as pH adjusting agent, buffer, antioxidants, antimicrobial preservatives, tonicity/osmotic modifying agents in an emulsion. In an emulsion of the current invention, the aqueous phase is the continuous phase in which the oil droplets are suspended and referred to as the dispersed phase. The aqueous phase can be separated from the oily phase by an ultrafiltration process, which allows for exchange of the aqueous phase with another aqueous solution with more desirable composition such as one with a lower ion content (as in Example 6). In certain embodiments, the preferred aqueous phase contains minimal amount of non-peptide and water-soluble ionic species.

As used herein, “body or near body temperature” is between 30° and 40° C.

As used herein, “chemical stability” or “chemically stable” means the state of a composition which retains no less than 75% of the initial glucagon after 1 year at 2-8° C. and 3-7 days at 37° C.

As used herein, “Critical Droplet Surface Area” is the minimum Total Droplet Surface Area in a nanoemulsion of the present invention that is required dissolve and stabilize 1 milligram of glucagon. According to the nanoemulsion of the present invention, the Critical Droplet Surface Area is estimated at about 3.0×10⁶ mm² in Total Droplet Surface Area. (Example 1). For example, to prepare a nanoemulsion of the present invention at a concentration of 1 mg/mL glucagon, each milliliter of such nanoemulsion must contain a Total Droplet Surface Area of about 3.0×10⁶ mm² As another example, to prepare a nanoemulsion of the present invention at a concentration of 0.5 mg/mL glucagon, each milliliter of such nanoemulsion must contain a Total Droplet Surface Area of at least about 1.5×10⁶ mm² In order to achieve the Critical Droplet Surface Area, a nanoemulsion must contain a sufficient amount of oily phase and the oily phase must be reduced to sufficiently small oil droplets. In certain embodiments, the oily phase concentration is about 20% and the mean droplet diameter is no greater than about 200 nm for every 1 mg of glucagon solubilized. In another embodiment, the oily phase concentration is between about 10% and 20% and the mean droplet diameter is between 100 and 200 nm for every 1 mg of glucagon solubilized. In a preferred embodiment, the oily phase concentration is about 15% and mean droplet diameter is less than 150 nm for every 1 mg of glucagon to be solubilized.

As used herein, the “Critical Ion Content Limit” is the maximum amount of non-peptide and water-soluble ions permitted in the nanoemulsion of the present invention in which glucagon remains chemically and physically stable. Above the Critical Ion Content Limit, the chemical and physical stability of glucagon may be adversely affected. The overall non-peptide and water-soluble ion concentration in an emulsion can be estimated by determining electrical conductivity. The value of Critical Ion Content Limit for a nanoemulsion of this invention is defined quantitatively as electrical conductivity equivalent to that measured for 0.12% sodium chloride solution in water, as determined under identical measurement conditions.

As used herein, “detergents” are water-soluble surfactants that can be used to dissolve and clean oily substances with water.

As used herein, “electrical conductivity” refers to a measurement of a material's ability to conduct an electric current. In water, electric conductivity is mediated by dissolved ions and is proportional to the total dissolved ion content. Electrical conductivity of a glucagon emulsion or solution is measured using an electrical conductivity meter in of μSiemens/cm or μS/cm units (Example 6). Electrical conductivity measurements are highly dependent on temperature, the individual conductivity meter, and the sample container. Therefore, comparative measurements must done using the same meter-container configuration and at the same temperature. For electrical conductivity measurements, solution standards with known NaCl concentrations are used to calibrate the conductivity meter. The dissolved ion concentration of an emulsion is then measured using the same conductivity meter-container configuration and is expressed in concentration units of sodium chloride solution (e.g., NaCl % w/v in water).

As used herein, an “emulsion” is a mixture of immiscible oily phase and aqueous phase. Typical emulsions are optically opaque and possess a finite stability, which are in contrast to the more transparent and stable nanoemulsions of the present invention.

As used herein, “filterable” means the ability of a liquid to pass through a filter membrane of a certain pore size such as 0.2-microns.

As used herein, the term “fine needle” includes a small-diameter, hollow hypodermic needle that is used with a syringe or pump for subcutaneous, intravenous, or other type of injection. The outer diameter of the needle is indicated by the needle gauge system. According to the Stubs Needle Gauge system, hypodermic needles in common medical use range from 7 (the largest) to 33 gauge (G)(the smallest). As used herein, “fine needle” therefore includes needles ranging from 21 to 33 G, preferably 25 G to 31 G and most preferably 27 G to 31 G.

As used herein, the term “injectable” means that a liquid meets the Acceptable Injectability Criterion as defined above.

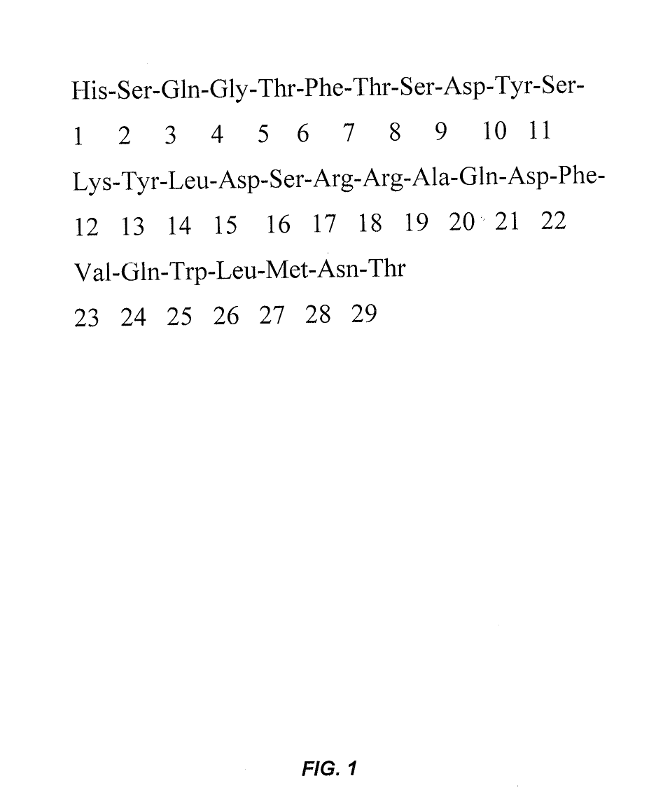

As used herein, “glucagon” refers to the full length peptide, glucagon, having the empirical formula of C₁₅₃H₂₂₅N₄₃O₄₉S, a molecular weight of 3,483 Da., and composed of a single-chain polypeptide containing 29 amino acid residues. The amino acid sequence of glucagon is shown in FIG. 1.

As used herein, “lecithin” is a mixture of phospholipids derived from a natural source. Injectable lecithin includes lecithin derived from egg or soybean, which have been purified and are substantially free from irritating, allergenic, inflammatory agents or agents that cause other deleterious biological reactions. For this invention, the preferred lecithins includes those that contain more than 75% phosphatidylcholine (PC), are insoluble in water and essentially free of lysolecithin (i.e., containing no more than 1-4% lysolecithin by weight). Examples of the preferred lecithins include but are not limited to lecithin products by the trade names of LIPOID S 75, LIPOID S 100, LIPOID E 80, and Phospholipon 90 G.

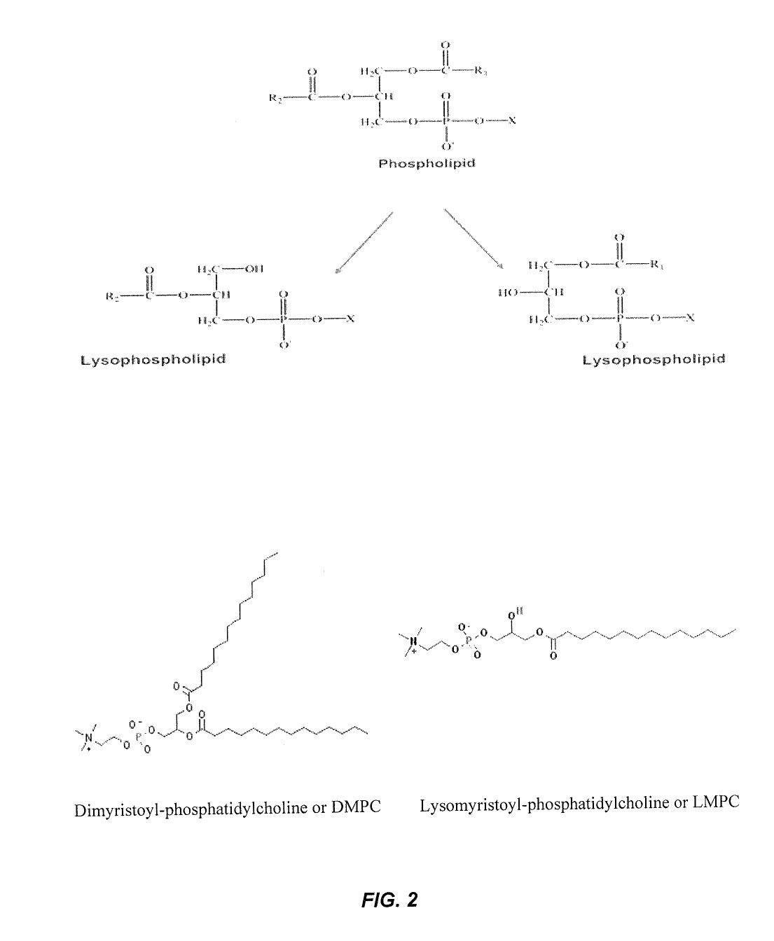

As used herein, “lysophospholipids,” which include lysophosphatidylcholines, as described below, are a class of chemical compounds which are derived from phospholipids as result of a partial hydrolysis of the phospholipid molecules, which removes one of the fatty acid groups (FIG. 2). Lysophospholipid are naturally occurring and can be synthetically produced. Unlike phospholipids, lysophospholipids have water-soluble surfactant properties and are capable of dissolving various lipophilic substances including nerve myelin and cell membranes. Lysophospholipids are known to cause hemolysis, and having such biological activities, are not safe for injection. Exemplary synthetic lysophospholipids include lysomyristoylphosphatidylcholine (LMPC), myristoyl lysophosphatidyl choline (LPCM), lysopalmitoylphosphatidylcholine (LPPC), as disclosed in US Patent Application 2011/0097386 and European Patent 1061947.

As used herein, “lysolecithins”, which are also known as “lysophosphatidylcholines”, are a subclass of lysophospholipids and are formed by the partial hydrolysis of lecithin, which removes one of the fatty acid groups. The hydrolysis is generally the result of the enzymatic action of phospholipase A2. Lysolecithins share much of the same water-solubility, detergent and hemolytic properties as lysophospholipids.

According to this invention, lysophospholipids (including lysolecithin) are undesirable and must be avoided in an injectable emulsion composition for glucagon. Such water-soluble surfactants can dissolve the emulsion oil droplets and disrupt the association of glucagon to the oil droplets resulting in reduced physical and chemical stability for glucagon. Moreover, lysophospholipids and lysolecithins are also known to be hemolytic and are toxic to humans if given by injection.

As used herein, “Mean Droplet Surface Area” is the surface area of droplets calculated using the measured mean radius of the droplets in an emulsion using the equation for the surface area of a sphere: A=4πr², where A is the Mean Droplet Surface Area, r is mean radius of the oil droplets of the emulsion, which is typically measured using a dynamic light scattering technique.

As used herein, “Mean Droplet Volume” is the volume of droplets calculated using the measured mean radius of the droplets in an emulsion using the equation for volume of a sphere: V=(4/3)πr³, where, V is the Mean Droplet Volume, r is mean radius of the oil droplets, which is typically measured using a dynamic light scattering technique.

As used herein, “mean radius” or “mean diameter” of the oil droplets” is a measured value of an emulsion using a dynamic light scattering or a laser diffraction method. The typical mean radius/diameter value is reported in nanometers.

As used herein, “medium chain oil” also known as “medium chain triglycerides” (“MCTs”) are medium-chain (6 to 12 carbons) fatty acid esters of glycerol. A MCT can be either derived from a natural source or made synthetically. Examples of the preferred MCT include MCT products by the trade names of CRODAMOL GTCC-PN, Miglyol 812 or Neobees M-5.

The term “metal ion chelating agent or chelator” includes a metal ion chelator that is safe to use in an injectable product. A metal ion chelator works by binding to a metal ion and thereby reduces the catalytic effect of that metal ion on the oxidation, hydrolysis or other degradation reactions. Metal chelators that are useful in this invention may include ethylenediaminetetraacetic acid (EDTA, edetate), glycine and citric acid and the respective salts or a mixture thereof.

As used herein, “nanoemulsion” is an oil-in-water emulsion having a Total Droplet Surface Area exceeding the Critical Droplet Surface Area. The Total Droplet Surface Area is a proportional to the concentration of the oily phase and inversely proportional to the droplet size. In order to exceed the Critical Droplet Surface Area, a nanoemulsion must contain the oily phase at a sufficiently high concentration in droplets of sufficient small size.

As used herein, “neutral pH” is in the range of 4 to 8, preferably 5.2 to 7.2. “Slightly acidic” means pH from about 2.5 to 4, preferably 2.7 to 3.5.

As used herein, “oil” refers to a single or mixture of triglycerides (e.g., triacylglycerols or triacylglycerides) that are liquid at body temperatures, e.g., about 37° C., and are pharmacologically acceptable for use in injectable drugs. A triglyceride is an ester derived from glycerol and three fatty esters. “Oil” can be derived from a natural source or synthetically made. Examples include vegetable oil, animal oil, medium chain oil for naturally sourced oil, or tricaprylin, triolein, or trimyristin for synthetic oil. For the present invention, the preferred oil is vegetable oil and the more preferred oil is medium chain oil.

As used herein, “oil-in-water emulsion” is an emulsion wherein the oily phase is in a form of small droplets (the dispersed phase), which are suspended or dispersed in the aqueous phase (continuous phase).

As used herein, “oily phase” refers to the water-immiscible phase of an emulsion comprising oil and phospholipid. The oily phase may also contain other lipophilic additives, including antioxidants and antimicrobial preservatives, etc. and glucagon as in the nanoemulsion of present invention. In certain embodiments, the oily phase exists in small oil droplets of size less than 200 nm, preferably less than 150 nm, and most preferably less than 100 nm in diameter.

As used herein, “osmolality” is the concentration of a solution in terms of milliosmoles of solutes per kilogram of solution. The preferred osmolality for the nanoemulsion of this invention is in the range of 280-700 mOsm/kg.

As used herein, “percent glucagon in the aqueous phase of an emulsion” is the measurement of the amount of glucagon in the aqueous phase over the total amount in the emulsion, i.e., percent glucagon in the aqueous phase of an emulsion is the amount of glucagon in the aqueous phase divided by amount of glucagon in the emulsion×100. The amount of glucagon in the aqueous phase of an emulsion can be determined by first separating the aqueous phase from the oily phase by ultrafiltration and then analyzing the glucagon concentration in the separated aqueous phase by an HPLC method (Example 2 & 10).

As used herein, the term “pH buffering agent” or “pH buffer salt” includes ionizable pH buffer salts such as phosphate, acetate, citrate, bicarbonate, and the like, with a counter-ion such as ammonium, sodium or potassium etc.

As used herein, “phospholipid” refers to any triesters of glycerol having two fatty acids and one phosphate ion which is covalently attached to a small organic molecule (such as choline, ethanolamine, glycerol, serine or nothing (noted as the “x” moiety in FIG. 2). Exemplary phospholipids hence include phosphatidylcholine (PC), phosphatidylethanolamine (PE), phosphatidylglycerol (PG), phosphatidylserine (PS), and phosphatidic acid (PA). The fatty acids generally have from about 10 to about 18 carbon atoms with varying degrees of saturation. For the present invention, “phospholipid” can refer to either a single phospholipid species or a mixture of several phospholipids. The phospholipids useful in the present invention can be obtained from natural sources or made synthetically. The naturally derived phospholipids are referred to as lecithin. Examples of synthetic phospholipids are: 1,2-dimyristoyl-sn-glycero-3-phosphocholine (DMPC), 1,2-distearoyl-sn-glycero-3-phosphoethanolamine (DSPE), 1,2-dimyristoyl-sn-glycero-3-phosphoglycerol, sodium salt (DMPG, Na), and 1,2-dipalmitoyl-sn-glycero-3-phospho-L-serine, sodium salt (DPPS, Na). In certain embodiments, the preferred synthetics phospholipids for the present invention are water insoluble phosphatidylcholine such as 1,2-dilauroyl-sn-glycero-3-phosphocholine (DLPC), 1,2-dimyristoyl-sn-glycero-3-phosphocholine (DMPC), 1,2-dipalmitoyl-sn-glycero-3-phosphocholine (DPPC), 1,2-distearoyl-sn-glycero-3-phosphocholine (DSPC), 1-palmitoyl-2-oleoyl-sn-glycero-3-phosphocholine (POPC) and a mixture thereof.

As used herein, “physical stability” or “physically stable” in reference to a liquid composition containing glucagon in which gelation, fibrils, or precipitates of glucagon are substantially absent and that the composition meets the “Acceptable Injectability Criterion” or that the composition is filterable through a 0.2 or 0.45-micron filter.

As used herein, “reducing agents” useful in this invention include, but are not limited to, ascorbic acid, ascorbate, ascorbyl palmitate, metabisulfite, propyl gallate, butylated hydroxyanisole, butylated hydroxytoluene, tocopherol, methionine, citric acid, citrate, a reducing sugar such as glucose, fructose, glyceraldehyde, galactose, lactose, maltose, a salt or a mixture thereof.

As used herein, the term “substantially free” means less than 1% of the total composition weight.

As used herein, a “sugar” refers to a carbohydrate additive(s) that can be added to a liquid composition to adjust the osmotic pressure or tonicity of an emulsion of the present invention. The sugars useful for this invention include, but are not limited to, monosaccharides, disaccharides, polysaccharides, propylene glycols, polyethylene glycols, glycerols, poly-ols, dextrins, cyclodextrins, starches, celluloses and cellulose derivatives, or mixtures thereof. For instance, in certain embodiments, the sugar is mannitol, sorbitol, xylitol, lactose, fructose, xylose, sucrose, trehalose, mannose, maltose, dextrose, dextran, or a mixture thereof. In other embodiments, the preferred sugar is glycerol or sucrose.

As used herein, “solution” refers to a clear, homogeneous mixture composed of only one phase.

As used herein, “surfactants” refers to compounds that lower the surface tension of a liquid or the interfacial tension between two liquids.

As used herein, the term “tonicity/osmotic modifying agent” comprises a pharmaceutical additive that can be added to an injectable pharmacologically active agent and be used to adjust osmolality. The tonicity/osmotic modifying agents useful in this invention include, but are not limited to, lactose, trehalose, sucrose, sorbitol, glycerol, mannitol, and mixtures thereof.

As used herein, the “Total Droplet Surface Area” is the total surface area of all oil droplets in a volume of nanoemulsion. This number is estimated by multiplying the Mean Droplet Surface Area by the Total Number of Oil Droplets.

As used herein, the “Total Number of Oil Droplets” in an emulsion is calculated by the dividing the total oily phase volume by the Mean Droplet Volume of the emulsion.

As used herein, “ultrafiltration” (UF) refers to a variety of membrane filtration techniques in which hydrostatic pressure forces a liquid against a semipermeable membrane. Suspended solids, oil droplets or solutes of high molecular weight or size are retained, while water and low molecular weight solutes, e.g., water-soluble ions, pass through the membrane. This separation process is commonly used in industry and research for purifying and concentrating macromolecular solutions, especially protein solutions. Ultrafiltration is not fundamentally different from diafiltration, microfiltration, nanofiltration or gas separation, except in terms of the size of the molecules it retains. Ultrafiltration is used in a tangential flow or dead-end mode. Ultrafiltration can be used to retain the glucagon-associated oil droplets while washing the aqueous phase to remove undesirable ions to will improve the chemical stability of glucagon.

As used herein, “vegetable oil” refers to oil derived from plant seeds or nuts. Exemplary vegetable oils include, but are not limited to, almond oil, borage oil, black currant seed oil, corn oil, safflower oil, soybean oil, sesame oil, cottonseed oil, peanut oil, olive oil, rapeseed oil, coconut oil, palm oil, canola oil, etc. Vegetable oils typically contain long-chain triglycerides, that are formed when three fatty acids (usually about 14 to about 22 carbons in length and having chains that with unsaturated bonds in varying numbers and locations, depending on the source of the oil) form ester bonds with the three hydroxyl groups on glycerol. In certain embodiments, vegetable oils of highly purified grade (also called “super refined”) are generally used to ensure safety and stability of pharmaceutical-grade oil-in-water emulsions.

As used herein, “water-soluble” describes a solid or liquid solute that can dissolve in water to form a homogeneous solution to an extent of no less than one weight part of solute in every ten weight parts of water.

As used herein, “water-soluble surfactants” are compounds help solubilize compounds to form a clear and one-phase aqueous solution by lowering the interfacial surface between water and another liquids or between water and a solid For example, lysolecithin is a water-soluble surfactant and lecithin is NOT a water-soluble surfactant.

As used herein, “tangential flow filtration”, which is also known as also known as “cross flow filtration”, is different from dead-end filtration where the feed is passed through the semipermeable membrane and the solids or emulsion droplets are trapped in the filter while the filtrate, such as the aqueous phase of an emulsion, is released at the other end. In tangential flow filtration, the majority of the feed flow travels tangentially across the surface of the filter, rather than perpendicularly into the filter. The principal advantage of tangential flow filtration is that the filter cake (which can clog the filter) is substantially washed away during the filtration process by the tangential flow, increasing the length of time that a filter unit can be operational. Tangential flow filtration is sometimes used interchangeably with “diafiltration”. A semipermeable membrane is a membrane that will allow certain small molecules or ions to pass through it by diffusion or forced diffusion while retaining solids or oil droplets of size greater than the membrane pore size, such as in the range of 10³ to 10⁶ Daltons. In certain embodiments, the preferred tangential flow filtration membrane pore size for the emulsion of the present invention is between 3K and 100K MWCO (molecular weight cut-off).

### III. Compositions of the Invention

The present invention provides an oil-in-water nanoemulsion composition comprising glucagon, an oily phase, and an aqueous phase. In some embodiments, the nanoemulsion composition comprises glucagon, an aqueous phase and oily phase, wherein the oily phase exists as nanometer-sized droplets with a mean diameter of less than 200 nm. In some embodiments, the present invention provides an oil-in-water nanoemulsion composition comprising glucagon, an oily phase, and an aqueous phase, wherein the glucagon is not aggregated and retains no less than 75% of its concentration after 3-7 days storage at 37° C., and wherein the oily phase is in the form of oil droplets having a mean diameter of less than about 200 nm.

The nanoemulsions can contain any suitable amount of glucagon. In general, the nanoemulsions contain glucagon in an amount of from about 0.01 to about 2 mg/mL. The nanoemulsion compositions of the present invention can contain, for example, 0.1 to 1.5 mg/mL of glucagon, or 0.5 to 1.5 mg/mL of glucagon. The nanoemulsion compositions can contain about 0.5 mg/mL, about 0.6 mg/mL, about 0.7 mg/mL, about 0.8 mg/mL, about 0.9 mg/mL, about 1.0 mg/mL, about 1.1 mg/mL, about 1.2 mg/mL, about 1.3 mg/mL, about 1.4 mg/mL and about 1.5 mg/mL of glucagon.

In some embodiments, the nanoemulsion compositions of the present invention contain 6.1 to 29% by weight, and more preferably 10% to 20% by weight, of an oily phase. The nanoemulsions can contain, for example, about 10%, about 11%, about 12%, about 13%, about 14%, about 15%, about 16%, about 17%, about 18%, about 19%, or about 20% by weight of an oily phase.

In some embodiments, the nanoemulsion composition includes an aqueous phase and an oily phase, wherein the oily phase comprises glucagon, oil, and phospholipid. In some embodiments, the oily phase contains phospholipid and oil.

In some embodiments, the nanoemulsion composition includes glucagon, oil, and phospholipid in an aqueous phase, wherein the glucagon remains in a non-aggregated, non-gelled or non-precipitated form.

The nanoemulsions can contain any suitable amount of oil. In general, the nanoemulsions contain oil in an amount of from about 0.1 to about 10% by weight. In some embodiments, the nanoemulsion compositions include 0.25 to 7.5% by weight, and more preferably 1 to 5% by weight, of oil. The nanoemulsions can include, for example, about 0.5%, about 0.75%, about 1.0%, about 1.25%, about 1.5%, about 1.75%, about 2%, about 2.5%, about 3%, about 4% or about 5% by weight of oil or a mixture of oils.

In general, the oils in the oily phase are triglycerides (e.g., triacylglycerols or triacylglycerides), alone or in combination, that are liquid at body temperatures and are pharmacologically acceptable for use in injectable drugs. The oils can be derived from a natural source or synthetically made. Examples of oils include, but are not limited to, vegetable oil, animal oil, medium chain oil for naturally sourced oil, or tricaprylin, triolein, or trimyristin for synthetic oil. Exemplary vegetable oils include, but are not limited to, almond oil, borage oil, black currant seed oil, corn oil, safflower oil, soybean oil, sesame oil, cottonseed oil, peanut oil, olive oil, rapeseed oil, coconut oil, palm oil, canola oil, etc. “medium chain oil” also known as “medium chain triglycerides” (“MCTs”) are medium-chain (6 to 12 carbons) fatty acid esters of glycerol. A MCT can be either derived from a natural source or made synthetically.

In some embodiments, the oil is a vegetable oil, such as sesame oil, castor oil, corn oil, or soybean oil. In some embodiments, the oil is a synthetic oil. In some embodiments, the oil is a medium chain oil. Some embodiments of the invention provide nanoemulsions wherein the oil is a medium chain oil, a vegetable oil, or a combination thereof.

The nanoemulsions can contain any suitable amount of phospholipid. In some embodiments, the nanoemulsion compositions of the present invention include 5 to 20% by weight, and more preferably 7.5 to 12.5% by weight, of a phospholipid. The nanoemulsions can contain, for example, about 7.5%, about 8%, about 8.5%, about 9%, about 9.5%, about 10%, about 10.5%, about 11%, about 11.5%, about 12% or about 12.5% by weight of a phospholipid or a mixture of phospholipids. Some embodiments of the invention provide nanoemulsions wherein the oily phase is between about 10% and 20% by weight of the nanoemulsion and the phospholipid concentration is more than the oil concentration.

Any suitable phospholipid can be used in the nanoemulsions of the invention. Suitable phospholipids include phosphatidylcholine (PC), phosphatidylethanolamine (PE), phosphatidylglycerol (PG), phosphatidylserine (PS), and phosphatidic acid (PA). The phospholipids useful in the present invention can be obtained from natural sources or made synthetically. The naturally derived phospholipids are referred to as lecithin. Synthetic phospholipids include, but are not limited to, DMPC; DSPE; DMPG, Na; DPPS, Na; DLPC; DMPC; DPPC; DSPC; and POPC; as described herein.

In some embodiments, the phospholipid is a lecithin. In some embodiments, the phospholipid is an egg lecithin or a soy lecithin. In some embodiments, the nanoemulsions include an aqueous phase and an oily phase, wherein the oily phase contains glucagon, medium chain oil, and lecithin derived from egg or soy bean. Some embodiments of the invention provide nanoemulsions made with egg or soy lecithin having a residual lysolecithin content of less than 5% of the lecithin weight.

In general, the nanoemulsions of the invention contain an aqueous phase in an amount of from about 70% to about 95% by weight. In some embodiments, the nanoemulsion compositions of the present invention include 71 to 92% by weight, and more preferably 80% to 90% by weight, of an aqueous phase. The nanoemulsions can include, for example, about 80%, about 81%, about 82%, about 83%, about 84%, about 85%, about 86%, about 87%, about 88%, about 89%, or about 90% by weight of an aqueous phase.

In some embodiments, each milliliter of the nanoemulsion composition contains 0.5 to 2 mg of glucagon, 5 to 100 mg of oil, 50 to 200 mg of phospholipid and 700 to 900 mg of an aqueous phase.

The nanoemulsions of the present invention can have any suitable pH. In general, the nanoemulsion of the present invention is at a pH between about 2.7 and about 8. More preferably, the pH is between about pH 2.7 and about 7.2. The pH can be, for example, about 2.7, 2.8, 2.9, 3.0, 3.1, 3.2, 3.3, 3.4, 3.5, 3.7, 3.9, 4.0, 4.2, 4.4, 4.6, 4.8, 5.0, 5.2, 5.4, 5.6, 5.8, 6.0, 6.2, 6.4, 6.6, 6.8, 7.0 or 7.2. The pH of the nanoemulsion can be achieved by the combining the components and adding an acid or base, and/or by the exchanging the nanoemulsion aqueous phase with another aqueous solution at the desired pH using ultrafiltration, dialysis, gel filtration, or another suitable technique. Some embodiments of the invention provide nanoemulsions wherein the pH is between about 2.7 to about 7.5. In some embodiments, the nanoemulsion has a neutral pH.

Surprisingly, stabilization of glucagon was found to depend on the size and surface area of the oil droplets in the nanoemulsions. Without wishing to be bound by any particular theory, it is believed that each droplet of a nanoemulsion composition is composed of an oil core having a surface coating which is rich in phospholipids which are bipolar molecules composed of a hydrophilic “head” and lipophilic “tail” (FIG. 2). The phospholipids on the oil droplet surface are believed to solubilize the glucagon in the emulsion by providing an environment where glucagon can exist in a non-aggregated form. Glucagon is insoluble and essentially not found in the aqueous phase (as shown in Example 10) and is also insoluble in pure oil, so it is unlikely that glucagon would preferentially partition into the oil droplet core. Rather, glucagon molecules are believed to be residing in the phospholipid layers at the oil droplet surface. As such, the droplets are likely coated with a layer(s) of glucagon molecules with the phospholipids. The surface of the droplets thus becomes a default localization for glucagon due to the thermodynamic exclusion of this peptide from both oily and aqueous phases. By localizing on the oil droplet surface, the glucagon molecules are less exposed to the hydrophobicity and hydrogen bond effects that otherwise would induce 13 sheets formation and/or aggregation, which would precipitate the glucagon from the emulsion.

Accordingly, the Total Droplet Surface Area becomes an important compositional feature of the nanoemulsion of present invention. The Total Droplet Surface Area can be estimated by multiplying the total number of droplets by the mean surface area of the individual droplets in the emulsion. At a fixed oily phase volume, the Total Droplet Surface Area is inversely proportional to the droplet size, i.e., the smaller the droplets, the greater Total Droplet Surface Area.

The total number of the droplets in a nanoemulsion of the invention can be calculated by dividing the total oily phase volume by the mean volume of the droplets. At a fixed oily phase volume, the total number of droplets is also inversely proportional to the droplet size, i.e., the smaller the droplets the greater number of droplets. TABLE 1 below illustrates the relationship of Total Droplet Surface Area and number of droplets on the mean droplet size at a fixed oily phase concentration.

TABLE 1
Calculation of Emulsion Droplet Surface Area

| | | | | | Meets/ |
|---|---|---|---|---|---|
| | | | | | Exceeds |
| | | | | | the |
| Oily | Mean | | | Total | Critical |
| phase | Droplet | Mean Droplet | Number of | Droplet | Droplet |
| conc. | Size | Surface Area | Droplets | Surface Area | Surface |
| (% v/v) | (nm, dia) | (mm²/droplet) | (#/mL) | (mm²/mL) | Area |
| 10 | 200 | 5.02E−07 | 2.99E+12 | 1.5E+06 | No |
| 10 | 150 | 2.83E−07 | 7.08E+12 | 2.0E+06 | No |
| 10 | 100 | 1.26E−07 | 2.39E+13 | 3.0E+06 | Yes |
| 10 | 80 | 8.04E−08 | 4.67E+13 | 3.8E+06 | Yes |
| 15 | 200 | 5.02E−07 | 4.48E+12 | 2.3E+06 | No |
| 15 | 150 | 2.83E−07 | 1.06E+13 | 3.0E+06 | Yes |
| 15 | 100 | 1.26E−07 | 3.58E+13 | 4.5E+06 | Yes |
| 15 | 80 | 8.04E−08 | 7.00E+13 | 5.6E+06 | Yes |
| 20 | 200 | 5.02E−07 | 5.97E+12 | 3.0E+06 | Yes |
| 20 | 150 | 2.83E−07 | 1.42E+13 | 4.0E+06 | Yes |
| 20 | 100 | 1.26E−07 | 4.78E+13 | 6.0E+06 | Yes |
| 20 | 80 | 8.04E−08 | 9.33E+13 | 7.5E+06 | Yes |
| 20 | 40 | 2.01E−08 | 7.46E+14 | 1.5E+07 | Yes |

Therefore, to create sufficient droplet surface area to solubilize glucagon, one can (1) increase the oily phase concentration to increase the number of droplets and/or (2) decrease the droplet size. However, the oily phase concentration can only be increased to an upper limit of about 20%, above which the emulsions become too viscous to inject through narrow gauge hypodermic needles. Droplet size appears to have a lower limit of about 50 to 100 nm mean diameter, below which is difficult to achieve and, if achieved, results in unstable droplets that will agglomerate and grow back in size. The oily phase concentration also has a lower limit. For example, if the oily phase concentration is reduced to less than 10%, the emulsion becomes incapable of solubilizing glucagon at 1 mg/mL. According to the present theory, to maintain aggregation-free glucagon at the desired concentration, a nanoemulsion of the present invention must have sufficiently highly oily phase concentration and sufficiently small droplet size—and these two features can be combined, represented and evaluated by the Total Droplet Surface Area value as described above.

Indeed, we found that about a Total Droplet Surface Area of about 3.0 E+06 mm² is needed to solubilize or de-aggregate each milligram of glucagon. Therefore, this value of 3.0 E+06 mm² is referred to as the “Critical Droplet Surface Area”. When a Total Droplet Surface Area is below the Critical Droplet Surface Area due to either insufficient oily phase and/or large droplets, the emulsion is likely to contain aggregated or insoluble glucagon (see Example 1).

Therefore, to solubilize glucagon in an aggregate-free form at a targeted glucagon concentration of 1 mg/mL, a nanoemulsion composition should have a Total Droplet Surface Area greater than or equal to the Critical Droplet Surface Area for one milliliter of glucagon in the nanoemulsion of the present invention.

Accordingly, some embodiments of the invention provide nanoemulsions containing glucagon and oil droplets, wherein the Total Droplet Surface Area is no less than 3.0 E+06 mm² for every 1 mg of glucagon. In some embodiments, the nanoemulsion composition comprises glucagon and oil droplets, wherein the oily phase concentration is no more than about 20% w/w and the mean droplet diameter is no greater than about 200 nm.

In some embodiments, the nanoemulsion composition comprises glucagon, an aqueous phase and an oily phase, wherein the oily phase is about 10 to 20% of the weight of the nanoemulsion and the Total Droplet Surface Area is equal to or greater than about 3.0 E+06 mm² for every milligram of glucagon.

In some embodiments, the nanoemulsion composition includes glucagon and oil droplets suspended in an aqueous phase, wherein the oily phase concentration is between 10% and 20% w/w and the average droplet diameter is between about 100 and 150 nm. The nanoemulsion composition may be used to solubilize glucagon at concentration up to approximately 1 mg glucagon per mL of nanoemulsion.

In some embodiments, the emulsion composition includes glucagon and oil droplets in an aqueous phase, wherein the oily phase concentration is about 15% w/w and average droplet diameter is less than 150 nm. The nanoemulsion composition may be used to solubilize glucagon at concentration up to approximately 1 mg glucagon per mL of nanoemulsion.

Surprisingly, it was found that glucagon is chemically stabilized and its various degradation pathways are suppressed to an acceptable level by the nanoemulsion composition of the present invention. Specifically, by having glucagon localized to the oil droplet surfaces, the emulsion composition demonstrated greatly improved chemical stability for glucagon compared to a solution composition wherein such oil droplets are absent.

The major degradation pathways for glucagon are aspartic acid cleavage at positions 9, 15, and 21 and glutaminyl deamidation at positions 3, 20 and 24, which leads to many degradation products (Kirsch, L. E., et al. 2000. Int. J. of Pharmaceutics. 203: 115-125). Both aspartic acid cleavage and glutaminyl deamidation involve nucleophilic attack of the ionized side-chain carboxylate on the protonated carbonyl carbon of the peptide bond to give a cyclic anhydride intermediate (“cyclic imide”) (Anjali, B., et al. 2005. J. Pharm. Sci. 94: 1912-1927). Certain flexibility in the peptide bond and side chain would be needed in order for the molecule to bend and form the 5-member ring of a cyclic imide.

Without wishing to be bound by any particular theory, it is believed that that by being localized to the oil droplet surface, glucagon molecules in the nanoemulsion compositions lose flexibility and are, therefore, less like to undergo the aspartic acid cleavage and glutaminyl deamidation. The resulting apparent chemical stabilization of glucagon by the emulsion droplets is a critical advantage for a commercially viable liquid glucagon drug product, since it would enable the composition to have the shelf life and stability required for widely-used and practical medical products (e.g., 1 year at 5° C. and 3-7 days at 37° C.). In the absence of such oil droplets of the present invention, glucagon degrades at a much faster rate. Example 12 and 13 show that glucagon is much more stable in the nanoemulsion compositions of the present invention than in an acidic solution (Example 12) or an aqueous solution composition disclosed in the prior art, both of which do not contain oil droplets (e.g., Novo Nordisk Glucagen Hypokit and U.S. Patent Application Publication No. 2011/0097386). To date, there are no known liquid compositions that provide a suitably stable (e.g., stable for 1 year at 5° C. and 3-7 days at 37° C.), aqueous, and ready-to-inject liquid glucagon drug product.

Accordingly, some embodiments of the invention provide nanoemulsions containing glucagon, oil, and phospholipid in an aqueous phase, wherein more than 50% of glucagon remains partitioned in or non-covalently associated with the oily phase. In some embodiments, more than 90% of the glucagon is non-covalently associated with the oily phase. In some embodiments, the Total Droplet Surface Area exceeds the Critical Droplet Surface Area for each milligram of glucagon contained in said nanoemulsion. In some embodiments, the Critical Droplet Surface Area is about 3.0×10⁶ mm² for each milligram of glucagon in the said nanoemulsion.

In the nanoemulsion of the present invention, glucagon can be further chemically stabilized by minimizing and/or removing non-peptide ions in the nanoemulsion compositions. Residual amounts of ions such as sodium, chloride, and acetate are commonly found in manufactured peptides as a result of the peptide purification process. Essentially, all commercially available peptides made by synthetic or DNA recombinant methods contain 1-10% by weight ions as counter ions or residual ions. These ionic species that accompany the peptide raw material are thus referred to as “non-peptide ions”.

Additional non-peptide ions may be introduced to a final glucagon formulation as a result of adding ionic stabilizers, solubilizers, pH buffer species and pH-adjusting agents. For example, the use of amino acid stabilizers (U.S. Pat. No. 5,652,216), phosphate or acetate as pH buffers (European Patent 1061947), lysolecithins as solubilizers (US Patent Application 2011/0097386), or hydronium and chloride ions as in commercial drug products (e.g., Glucagen Hypokit (glucagon hydrochloride) kit manufactured by Novo Nordisk and the Glucagon for Injection (rDNA Origin) produced by Eli Lilly and Company). Such non-peptide ions are usually soluble in water or in the aqueous phase of an emulsion and their overall concentration is quantitated by the electrical conductivity of the final composition. Since previously known glucagon compositions contain ionic glucagon in an aqueous solution and rely on ionic solubilizers, the resultant final composition inevitably contains a higher overall ion concentration or has a notably higher electrical conductivity, compared to the nanoemulsion of the present invention.

Surprisingly, the present inventors found that non-peptide ions are detrimental to glucagon's chemical stability (Example 6). Therefore, whenever possible, it is very desirable to avoid the use of ionic excipients (e.g., sodium, chloride, solubilizers, pH buffers, etc.) and/or to remove any counter-ions and/or non-peptide ions contained in the glucagon raw material. Thus, the nanoemulsion composition of the present invention avoids the use of ion-contributing excipients such as ionic pH buffers, pH-adjusting agents, tonicity modifiers, or surfactants.

In addition, since counter-ions and/or residual ions preferentially exist in the aqueous phase of the nanoemulsion, these ions can be removed by exchanging the nanoemulsion's aqueous phase with an ion-free aqueous phase using an ultrafiltration separation process such as dialysis, diafiltration, or tangential flow filtration (Example 6). After such processing, the concentration of counter-ions and/or residual ions in the nanoemulsion is greatly reduced.

In some embodiments, the nanoemulsion composition of the present invention comprises glucagon and oil droplets in an aqueous phase, wherein the electrical conductivity of the nanoemulsion is below what is measured for a 0.15% NaCl solution in water under the same conditions.

In some embodiments, the nanoemulsion composition comprises glucagon and oil droplets in an aqueous phase, wherein the total non-peptide ion content is less than 0.4% of the total nanoemulsion weight.

In some embodiments, the nanoemulsion composition comprises glucagon and oil droplets in an aqueous phase, wherein the nanoemulsion has been subjected to a membrane filtration process to remove the non-peptide ions by exchanging said aqueous phase with an ion-free or low ion aqueous solution.

Some embodiments of the present invention provide nanoemulsions wherein the ion content is less than the Critical Ion Content Limit, which is equal to electrical conductivity of a 0.12% w/w sodium chloride solution.

In aqueous vehicles, glucagon is believed to undergo oxidation at methionine 27. Thus, the presence of certain antioxidants can further improve glucagon stability. In some embodiments, the nanoemulsions as described above further contain at least one antioxidant selected from the group consisting of EDTA, methionine, fructose, dextrose, cysteine, glutathione or salt or a combination thereof. In some embodiments, the nanoemulsion contains an antioxidant selected from a group comprising methionine, cysteine, dextrose, fructose, lactose, and a salt of edetate (EDTA). In some embodiments, the nanoemulsion composition contains methionine, EDTA, or a combination thereof. In some embodiments, the nanoemulsion contains about 0.1 to 1% methionine, about 0.001 to 0.01% EDTA, or a combination thereof.

The nanoemulsions can optionally contain an anti-microbial preservative. Such preservatives can enable the composition to provide multiple doses from the same vial (multiple-dose vial format). In some embodiments, the nanoemulsion composition contains an anti-microbial preservative selected from a cresol, paraben, phenol, benzalkonium chloride, benzoic acid, benzoate, benzyl alcohol, chlorobutanol, thimerosal, sorbic acid, sorbate, EDTA or a combination thereof. In some embodiments, the nanoemulsion composition contains an anti-microbial preservative selected from EDTA, benzyl alcohol, a paraben, sodium metabisulfite, a cresol, or a salt and combinations thereof. In some embodiments, the nanoemulsion composition contains cresol as an anti-microbial preservative. Any suitable amount of anti-microbial preservative can be included in the nanoemulsions.

In some embodiments, the aqueous phase contains an antioxidant, a metal ion chelator, an antimicrobial preservative, a non-ionic sugar and water. In some embodiments, the emulsion is substantially-free of any water-soluble surfactants such as lysophospholipid, lysolecithin, SDS, CTAB, or cyclodextrin.

In general, the present invention provides nanoemulsion compositions containing glucagon which satisfy the Acceptable Injectability Criterion. In some embodiments, the nanoemulsion composition contains glucagon, oil, and phospholipid in an aqueous vehicle, and the nanoemulsion is readily injectable through a fine needle.

In some embodiments, the present invention provides nanoemulsion compositions containing glucagon which can be filtered through a 0.2- or 0.45-micron pore membrane and to permit sterilization by filtration, thus eliminating the need for an aseptic process or terminal sterilization using heat or radiation. In some embodiments, the nanoemulsion is filterable through a 0.2-micron filter.

The nanoemulsion compositions of the invention are characterized by advantageous stability. In some embodiments, the nanoemulsion composition is physically stable and does not contain aggregated, gelled or precipitated glucagon after the nanoemulsion has been stored for 1 year at 5° C.

In some embodiments, the emulsion composition is physically stable and does not contain aggregated, gelled or precipitated glucagon after the nanoemulsion has been stored for 3-7 days at 30-37° C.

In some embodiments, the emulsion composition is physically stable and does not contain aggregated, gelled or precipitated glucagon after the nanoemulsion has been stored for 1 year and after said nanoemulsion has been stored at 37° C. for 3-7 days.

In some embodiments, the nanoemulsion composition comprises glucagon and oil droplets in an aqueous phase, wherein more than 50% of the glucagon is adherent to or non-covalently associated with the oil droplets and the glucagon is chemically stable for 1 year at ° C.

In some embodiments, the nanoemulsion composition comprises glucagon and oil droplets in an aqueous phase, wherein more than 50% of the glucagon is adherent to or non-covalently associated with the oil droplets and the glucagon is chemically stable for 3-7 days at 37° C.

In some embodiments, the nanoemulsion composition comprises glucagon and oil droplets in an aqueous phase, wherein more than 50% of the glucagon is adherent to or non-covalently associated with the oil droplets and the glucagon is chemically stable for 3-7 days at 37° C. and for 1 year at 5° C.

In some embodiments, the emulsion composition comprises glucagon and oil droplets in an aqueous phase, wherein more than 50% of the glucagon is adherent to or non-covalently associated with the oil droplets. In the emulsion, the glucagon is physically stable for 3-7 days at 37° C. and for 1 year at 5° C.

In some embodiments, the present invention provides nanoemulsion compositions containing glucagon which are physically stable for at least 1 year at 2-8° C. or for 3-7 days at body or near body temperature (30-37° C.).

In some embodiments, the present invention provides nanoemulsion compositions containing glucagon which are chemically stable for at least 1 year at 2-8° C. or for 3-7 days at body or near body temperature (30-37° C.).

In some embodiments, the present invention provides nanoemulsion compositions containing glucagon which are physically and chemically stable for at least 1 year at 2-8° C. or for 3-7 days at body or near body temperature (30-37° C.).

The nanoemulsion compositions of the present invention can be lyophilized to further improve their physical and chemical stability. A lyophilized composition be reconstituted to form a liquid nanoemulsion prior to injection. The lyophilized nanoemulsions are provided as a dry mass “lyophile cake” in vials or syringes and are intended to be stable when stored at room temperature for at least one year. Before use, the lyophilized compositions are reconstituted with water, for example, to re-form a nanoemulsion with the same physical or chemical stability as the aforementioned liquid nanoemulsion compositions.

Accordingly, some embodiments of the invention provide a lyophilized dry composition containing glucagon, a phospholipid, a medium chain oil, and a sugar, wherein, upon mixing with water, the lyophilized dry composition forms a nanoemulsion as described above. In some embodiments, the lyophilized dry composition contains glucagon, a phospholipid, a medium chain oil, and a sugar, whereupon mixing with water, said lyophilized dry composition forms a nanoemulsion, wherein glucagon is not aggregated and retains no less than 75% of its concentration after 3-7 days storage at 37° C., and wherein: no less than 50% of the glucagon is non-covalently associated with the oily phase; the Total Droplet Surface Area exceeds the Critical Droplet Surface Area or 3.0×10⁶ mm² of droplet surface area for each milligram of glucagon contained in said nanoemulsion; the electrical conductivity of the said nanoemulsion is no more than the electrical conductivity of a 0.15% w/w sodium chloride solution in water; and the pH is between about 2.7 and about 7.5.

In some embodiments, the nanoemulsion composition is not lyophilized. In some embodiments, the nanoemulsion is ready-to-inject. For example, the emulsion composition can be stored at 5° C. as a translucent, uniform liquid which is suitable as a ready-for-injection format for administration via subcutaneous, intramuscular, or intravenous route or by a bi-hormonal insulin/glucagon pump. In some embodiments, the emulsion composition (or a reconstituted emulsion) is delivered via subcutaneous, intramuscular, or intravenous route by manual delivery or by a bi-hormonal insulin/glucagon pump as a treatment of a medical condition. In some embodiments, a lyophilized composition is mixed with water before injection. In some embodiments, the nanoemulsions and lyophilized composition are provided in vials or syringes.

In some embodiments, the present invention provides oil-in-water nanoemulsion compositions containing:

0.1 to 1.5 mg/mL glucagon;
0.5 to 7.5% by weight of oil;
5 to 20% by weight of one or more phospholipids; and
an aqueous phase to make up the remaining weight, wherein (a) said nanoemulsion contains oil droplets with a mean diameter of less than about 200 nm and (b) no less than 50% of the glucagon is non-covalently associated with the droplets.

In some embodiments, the present invention provides oil-in-water nanoemulsion compositions, comprising:

0.1 to 1.5 mg/mL glucagon;
0.5 to 7.5% by weight of oil;
5 to 20% by weight of one or more phospholipids; and
an aqueous phase to make up the remaining weight, wherein (a) said nanoemulsion contains oil droplets with a mean diameter of less than about 200 nm, (b) no less than 50% of the glucagon is associated non-covalently with the droplets and (c) the total non-peptide and water soluble ions do not exceed the Critical Ion Content Limit.

In some embodiments, the present invention provides an oil-in-water nanoemulsion compositions, comprising:

0.1 to 1.5 mg/mL glucagon;
0.5 to 7.5% by weight of oil;
5 to 20% by weight of one or more phospholipids; and
an aqueous phase to make up the remaining weight, wherein (a) said nanoemulsion contains oil droplets with a mean diameter of less than about 200 nm, (b) no less than 50% of the glucagon is associated non-covalently with the droplets, (c) the total non-peptide and water soluble ion content in the emulsion does not exceed the Critical Ion Content Limit, and (d) has a pH between 2.7 to 7.5.

In some embodiments, the nanoemulsion composition comprises 0.5 to 1.5 mg/mL glucagon, 0.5 to 10% by weight medium chain oil, 5 to 15% by weight egg lecithin, 0.1 to 1% by weight methionine, 0.0025 to 0.1% by weight EDTA disodium dehydrate, wherein such composition the pH is at between 2.7 and 7.

In some embodiments, the nanoemulsion composition comprises about 1 mg/mL glucagon, 0.5 to 5% medium chain oil, 10% egg lecithin, 0.3 to 1% methionine, 0.005% EDTA disodium dehydrate, wherein such composition the pH of the emulsion is between 2.7 and 7.

In some embodiments, the nanoemulsion composition comprises about 1 mg/mL glucagon, 0.5 to 5% by weight medium chain oil, 10% by weight egg lecithin, 0.3 to 1% by weight methionine, 0.005% by weight EDTA disodium dehydrate, wherein such composition the oily phase exists in oil droplets having a mean diameter less than 200 nm.

In some embodiments, the nanoemulsion composition comprises about 1 mg/mL glucagon, 1 to 5% by weight medium chain oil, 10% by weight egg lecithin, 0.3% by weight methionine, 0.005% by weight EDTA disodium dehydrate, wherein such composition the Total Droplet Surface Area exceeds the Critical Droplet Surface Area.

In some embodiments, the nanoemulsion composition comprises about 1 mg/mL glucagon, 0.5 to 5% medium chain oil, 10% egg lecithin, 0.3% methionine, 0.005% EDTA disodium dehydrate, wherein such composition at least 50% of the glucagon is in the oily phase.

In some embodiments, the nanoemulsion composition comprises about 1 mg/mL glucagon, 0.5 to 5% medium chain oil, 10% egg lecithin, 0.3 to 1% methionine, 0.005% EDTA disodium dehydrate, wherein the electrical conductivity of said nanoemulsion is no greater than the electrical conductivity of a 0.15% NaCl solution in water.

In some embodiments, the nanoemulsion compositions of the present invention comprise about 1 mg/mL glucagon, about 0.5 to 5% by weight a medium chain oil, about 10% by weight an egg lecithin, about 10% by weight sucrose, about 0.3% methionine, about 0.0055% EDTA disodium dehydrate and sufficient water to make up the rest of the total weight of the composition.

In some embodiments, the nanoemulsion compositions of the present invention contain about 1 mg/mL glucagon, about 0.5 to 5% by weight a medium chain oil, about 10% by weight an egg lecithin, about 10% by weight sucrose, about 0.3% by weight methionine, about 0.0055% by weight EDTA disodium dehydrate and sufficient water to make up the rest of the weight of the composition, wherein (a) said nanoemulsion contains oil droplets of a mean diameter of less than about 200 nm, (b) no less than 50% of the glucagon is associated non-covalently with the droplets.

In some embodiments, the nanoemulsion compositions of the present invention comprise about 1 mg/mL glucagon, about 0.5 to 5% by weight a medium chain oil, about 10% by weight an egg lecithin, about 10% by weight sucrose, about 0.3% by weight methionine, about 0.0055% by weight EDTA disodium dehydrate and sufficient water to make up the rest of the weight of the composition, wherein (a) said nanoemulsion contains oil droplets of a mean diameter of less than about 200 nm, (b) no less than 50% of the glucagon is associated non-covalently with the droplets and (c) the overall non-peptide and water-soluble ion content does not exceed the Critical Ion Content Limit.

In some embodiments, the nanoemulsion compositions of the present invention comprise about 1 mg/mL glucagon, about 0.5 to 5% by weight a medium chain oil, about 10% by weight an egg lecithin, about 10% by weight sucrose, about 0.3% by weight methionine, about 0.0055% by weight EDTA disodium dehydrate and sufficient water to make up the rest of the weight of the composition, wherein (a) said nanoemulsion contains oil droplets of a mean diameter of less than about 200 nm, (b) no less than 50% of the glucagon is associated non-covalently with the droplets and (c) the overall non-peptide and water-soluble ion content does not exceed the Critical Ion Content Limit and (d) is at a neutral pH.

In some embodiments, the nanoemulsion compositions of the present invention comprise about 1 mg/mL glucagon, about 0.5 to 5% by weight a medium chain oil, about 10% by weight an egg lecithin, about 10% by weight sucrose, about 0.3% by weight methionine, about 0.0055% by weight EDTA disodium dehydrate and sufficient water to make up the rest of the weight of the composition, wherein the Total Droplet Surface Area exceeds the Critical Droplet Surface Area.

In some embodiments, the nanoemulsion compositions of the present invention comprise about 1 mg/mL glucagon, about 0.5 to 5% by weight a medium chain oil, about 10% by weight an egg lecithin, about 10% by weight sucrose, about 0.3% by weight methionine, about 0.0055% by weight EDTA disodium dehydrate and sufficient water to make up the rest of the weight of the composition, wherein said nanoemulsion is substantially free of lysophospholipid or lysolecithin.

In yet another embodiment, the nanoemulsion of the present invention is formed by mixing a nanoemulsion vehicle with a dry glucagon formulation at a predetermined ratio prior to dosing by injection or by an insulin pump. A glucagon nanoemulsion prepared by this method is referred to as an “admixture” or “admix” and has the same composition as the pre-fabricated glucagon nanoemulsions of the present invention.

The nanoemulsion vehicles used to make the admixtures do not contain glucagon, but they may comprise the same inactive components as the pre-fabricated glucagon nanoemulsions of the present invention. The nanoemulsion vehicle used to make the admixtures may also have the similar physical properties and be prepared by the similar procedure as the as the pre-fabricated glucagon nanoemulsions of the present invention.

The dry glucagon formulation used to make the admixture may be a purified glucagon drug substance such as the Glucagon, USP, a dry mixture of glucagon drug substance with other pharmaceutically acceptable and injectable inactive ingredients, or a lyophilized formulation containing glucagon. In a preferred aspect, the dry glucagon formulation for making an admixture is the lyophilized glucagon formulation provided in the commercially available glucagon rescue kits (GlucaGen Hypokit (glucagon hydrochloride) from Novo Nordisk and Glucagon for Injection (rDNA origin) from Eli Lilly and Company).

To form the admixture, the dry glucagon formulation is mixed with and dissolved in the nanoemulsion vehicle upon gentle manual mixing. The preferred time of mixing or dissolution is less than 1 minute. The mixing procedure may involve adding the nanoemulsion vehicle to the dry glucagon formulation or vice versa at a pre-determined mixing ratio. The mixing ratio will allow formation of an admixture to contain a therapeutically sufficient amount of glucagon (e.g. 0.1 to 10 mg per vial) and pharmaceutically acceptable amounts of inactive ingredients. The admixture will have the same physical features as the pre-fabricated glucagon nanoemulsions of the present invention.

A preferred nanoemulsion vehicle used for admixture is sufficiently stable for long-term storage (e.g. 1-4 years) and meets the safety requirements as a drug diluent for injection.

A preferred admixture is more stable than the glucagon solutions prepared by reconstituting the glucagon lyophiles with the accompanied diluents in the Novo's GlucaGen Hypokit or the Lilly's Glucagon for Injection kit. The diluent provided in the Novo's and Lilly's kits are water and diluted hydrochloric acid, respectively.

In one aspect, the nanoemulsion vehicle is used to replace the diluent in Novo's or Lilly's Glucagon kit for hypoglycemia rescue. The nanoemulsion vehicle may be used at the same or different volumes as the diluents in the Novo or Lilly's kit. In another aspect, the admixture is sufficiently stable so it can be stored as a liquid in a refrigerator, at room temperature or body temperature for several days or months.

In one aspect, the admixture may be filled in a vial with a single dose or multiple doses. The content of the vial can be withdrawn and injected with needles and syringes. In another aspect, the admixture is filled in a syringe or auto-injector; both are ready-to-use. In another aspect, the admixture is stable for use in a subcutaneous infusion pump such as an insulin pump. A preferred admixture is stable and pump-able for 0.5 to 4 weeks after being loaded into the pump and used under the normal insulin pump use conditions, i.e. near body temperature with certain agitation.

### IV. Methods of Making Nanoemulsions and Lyophilized Compositions

In one embodiment, the present invention provides a method for preparing a nanoemulsion composition comprising glucagon. The method includes:
Step 1. combining, mixing, and dissolving a metal ion chelator (e.g., EDTA disodium dehydrate), antioxidant (e.g., methionine), sugar (e.g., sucrose), and glucagon in water (e.g., Water For Injection) or a diluted acid (e.g. 1-10 mM HCl) to form an aqueous solution;
Step 2. combining oil (e.g., a medium chain oil), phospholipid (e.g., egg lecithin) and a calculated amount of aqueous phase up to the targeted total weight or volume, and mixing vigorously until all solids are dissolved or dispersed to form a primary emulsion;
Step 3. passing the primary emulsion through a homogenizer to obtain a nanoemulsion with an average droplet diameter of less than about 200 nm;
Step 4. passing the nanoemulsion through a 0.2-micron filter to sterilize; and
Step 5. filling the filtered nanoemulsion into vials or syringes.

In some embodiments, glucagon, oil and phospholipid are first combined and dissolved in a volatile solvent such as ethanol to form a clear solution. The solvent is removed by drying with heat, vacuum or a stream of inert gas, such as nitrogen, to form a dry oily phase. The oily phase is then mixed with the aqueous phase and carried through Steps 2 to 5 above.

In some embodiments, no additional ionic additive is added at any of the above steps to avoid increasing the ion concentration in the nanoemulsion to above the Critical Ion Content Limit. This includes avoidance of any pH buffering salt. This also requires that the total counter ions content in the glucagon raw material must not be greater than about 5% of the total weight of the glucagon raw material.

Should the composition exceed the Critical Ion Content Limit, the primary emulsion (produced at Step 2) or the nanoemulsion (produced at Step 3) can be optionally subject to an ultrafiltration process to reduce the ion content to less than the Critical Ion Content Limit. The ultrafiltration process can take place after Step 2 or Step 3.

In some embodiments, an ultrafiltration process is applied to the primary emulsion or nanoemulsion to replace the emulsion aqueous phase with its higher content of extraneous counter ions from the glucagon raw material and other ingredients, with a new ion-free or low-ion containing aqueous phase to reduce the aqueous phase ion content to below the Critical Ion Content Limit. A typical volume exchange of about 1×, 2×, 3×, 4× or 5× of the aqueous phase is needed to deplete the dissolved and unwanted ions. For a small volume emulsion, a diafiltration device such as an Amicon Stirred Cell can be used to remove the unwanted ions to below the Critical Ion Content Limit. On a large scale, a tangential flow filtration (TFF) apparatus such as the Millipore Pellicon TFF cassette (Millipore Corp.) can be used. A semipermeable membrane with a MWCO of about 3K, 10K, 30K, 50K or 100K can be used to retain the oil droplets and associated glucagon while allowing passage of the dissolved and unwanted ions.

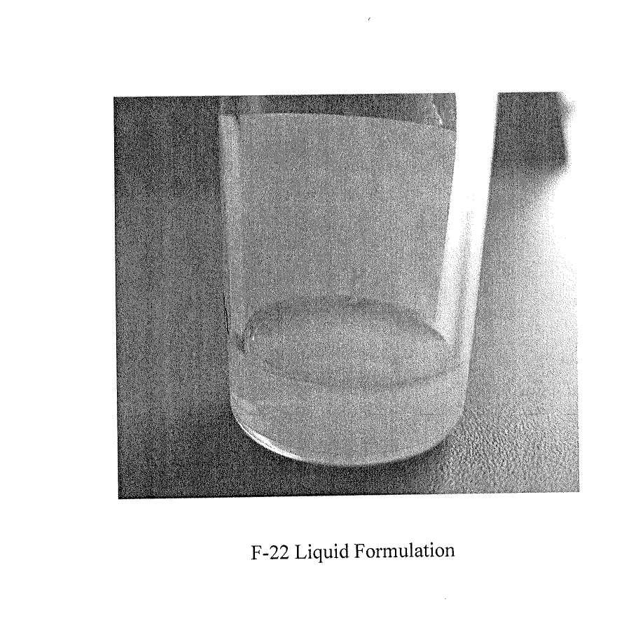

In some embodiments, a high-shear, high-energy or high-pressure homogenizer (such a microfluidizer available from Microfluidics International Corporation) is used to convert the primary emulsion to a nanoemulsion by reducing the oil droplet diameter in the primary emulsion from greater than 500 nm to less than about 200 nm, preferable less than about 150 nm and most preferably less than about 100 nm in diameter. The reduction of oil droplet size greatly reduces viscosity, increases the injectability of the nanoemulsion, and creates sufficient droplet surface area to exceed the Critical Droplet Surface Area, which is required for the physical and chemical stability in the glucagon nanoemulsion. High-pressure homogenization causes profound changes in the emulsion properties. For example, a primary emulsion of the present invention is a generally white, opaque, thick and cream-like liquid, which is not filterable through a 0.2-micron filter and is, therefore, not suitable for injection. The nanoemulsion, on the other hand, is semi-transparent, silky smooth, thin, and water-like liquid with a remarkably reduced viscosity (FIG. 5). The nanoemulsion can be filtered easily through a 0.2-micron filter (Examples 1 and 6).

Accordingly, the nanoemulsion is filtered through a sterile 0.2- or 0.45-micron filter membrane in some embodiments of the invention, sterilizing the composition prior to filling into vials or syringes. This filterability through a sterilizing filter is highly desirable since there is no other way to sterilize glucagon in the liquid form. Other common sterilization methods including gamma irradiation, high temperature treatment, sterilizing gas treatment (e.g., ethylene oxide) or UV light exposure can cause unacceptable damage to the chemical integrity of glucagon. Filtration is the most gentle and convenient method to sterilize a liquid composition, and this method of sterilization is made feasible by the nanoemulsion of the present invention.

The fact that the nanoemulsion is easily filterable through a 0.2- or a 0.45-micron filter indicates an absence of aggregated, gelled or precipitated glucagon. Furthermore, the nanoemulsion of the present invention remains filterable with average droplet size less than 200 nm after 3-7 days at the body or near body temperature (Example 8). In contrast, the Glucagon for Injection product from Eli Lilly and Company rapidly gels and is not filterable shortly after reconstitution. A lysophospholipid-based glucagon composition (US Patent Application 2011/0097386 or European Patent 1061947) is also not filterable after being at or near body temperature for 7 days (Example 13).

As such, some embodiments of the invention provide a process for preparing any of the nanoemulsions described herein. The process includes:
(a) combining glucagon and an aqueous phase
(b) adding phospholipid and oil
(c) mixing and homogenizing to form a nanoemulsion having an average droplet size of no more than 200 nm in diameter, and
(d) passing said nanoemulsion through a 0.2-micron filter.

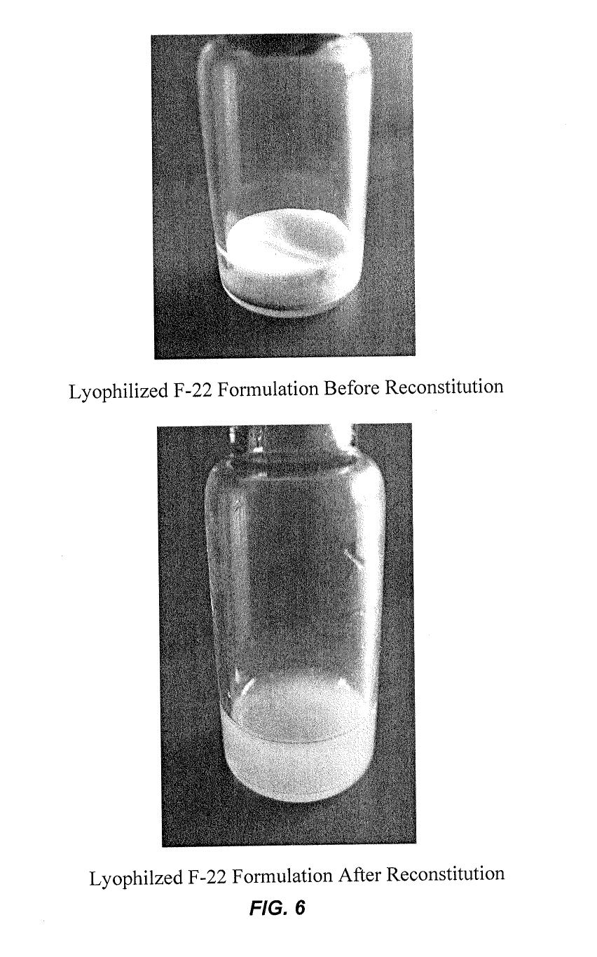

In some embodiments, the nanoemulsion of the present invention is lyophilized after Step 5 in the method for preparation described above to further improve the physical and chemical stability. The lyophilized nanoemulsion is provided as a dry mass “lyophile cake” in a vial (FIG. 6) or syringe and is intended to be stable at room temperature for at least one year. Before use, it is reconstituted with water to re-form the nanoemulsion having the same physical or chemical stability as the aforementioned liquid nanoemulsion compositions (FIG. 6).

Accordingly, some embodiments of the invention provide a method of making the dry compositions described herein. The method includes: combining glucagon and an aqueous phase
(a) adding phospholipid and oil
(b) mixing and homogenizing to form a nanoemulsion having an average droplet size of no more than 200 nm in diameter,
(c) passing said nanoemulsion through a 0.2-micron filter, and
(d) lyophilizing the nanoemulsion.

### V. Methods of Use and Administration

In another aspect, the invention provides a method of treating a patient in need of glucagon. The method includes administering to the patient any of the nanoemulsions and reconstituted lyophilized compositions described above. In some embodiments, the nanoemulsion of the present invention is provided in a pre-filled syringe with attached hypodermic needle attached and is ready for injection. This feature is particularly desirable for emergency hypoglycemia rescue. A typical dose used to reverse severe hypoglycemia is 1 mL of a 1 mg/mL nanoemulsion.

In some embodiments, the nanoemulsion of the present invention is administered via an intravenous, intramuscular or subcutaneous injection. In some embodiments, the glucagon is administered from a pump or from a syringe via a needle through a subcutaneous, intramuscular or intravenous route.

In some embodiments, the nanoemulsion of the present invention is filled in a cartridge (reservoir) or a vial and fitted to a pump and its liquid content is delivered by subcutaneous infusion from the pump in the treatment of diabetic conditions. To use glucagon in a pump, pump cartridges (obtained prefilled from a manufacturer or self-filled by the end user) are loaded into the pump device. At the end of term of use (e.g., 2-7 days), the remaining glucagon nanoemulsion is discarded and fresh glucagon nanoemulsion provided to the pump. The dose of glucagon delivered by subcutaneous infusion will be determined by the needs of the patient. In studies of a prototype insulin-glucagon bihormonal pump, the amount of glucagon used to achieve blood glucose control over a 24 hr period was reported to be 0.120 to 0.377 mg for an adult (El-Khatib, et al. 2010. Science Transl. Med. 2:27ra27).

In some embodiments, the nanoemulsion of the present invention contains an antimicrobial preservative and is filled in a vial or injection device (i.e., a pre-filled syringe or a vial in an autoinjector, among other configurations). The vial/syringe contains sufficient quantity for multiple doses and said content may be dosed to patients in multiple injections. Each time, a small and varying volume of the content is injected. This multiple-dose and variable dose feature would be particularly desirable for certain radiology procedures to inhibit gastrointestinal motility during radiology examination, for which a lower dose of glucagon is used. The addition of the antimicrobial preservative in the nanoemulsion prevents potential microbial growth after multiple punctures of the vial to remove multiple small doses or multiple injections using the same prefilled syringe. An antimicrobial preservative is also desirable for the bi-hormonal pump application, which exposes the nanoemulsion near body temperature for several days.

In some embodiments, the nanoemulsion of the present invention is provided as a dry mass (“lyophile cake”) in a vial or syringe and is intended to be stored at room temperature for at least one year. Before use, the lyophile cake is reconstituted with water to re-form a nanoemulsion having the same previous physical or chemical stability and which can be used in the same manner as the aforementioned liquid nanoemulsion compositions.

### VI. Examples

The present invention will be further understood by reference to the following non-limiting examples.

### Example 1

### Preparation of Physically Stable Nanoemulsions Containing Glucagon

The emulsion compositions in Table 2 were prepared to solubilize glucagon. Each composition was coded with a unique “F” number.

TABLE 2
Composition (% wt)

| Component | F-1 | F-2 | F-3 | F-4 |
|---|---|---|---|---|
| Glucagon (peptide content = 91.8%, counter- | 0.1* | 0.1 | 0.1 | 0.1 |
| ions = 3.7%) | | | | |
| Egg lecithin (Phosphatidylcholine content no | 10 | 5 | 10 | 5 |
| less than 80%) | | | | |
| Medium chain oil | 10 | 10 | 5 | 5 |
| Aqueous phase** | 79.9 | 85.9 | 84.9 | 89.9 |
| Oily phase (=glucagon + lecithin + oil wt) | 20.1 | 15.1 | 15.1 | 10.1 |
| Total | 100 | 100 | 100 | 100 |

*Approximately 1 mg/mL glucagon
**Aqueous phase contains 10% by wt sucrose and 0.0055% by wt EDTA disodium dehydrate in deionized water (DI-water). EDTA disodium dehydrate was added as an antimicrobial preservative.

The aqueous phase was prepared by weighing out 10 g sucrose and 5.5 mg EDTA disodium dihydrate, adding DI-water to 100 g, and dissolving all solids.

Emulsions were prepared by:
1. weighing out egg lecithin, medium chain oil, and glucagon in a plastic vial;
2. adding 50% batch size of ethanol;
3. mixing to dissolve all solids;
4. vacuum drying to remove the ethanol using a SpeedVac to a residual ethanol content of <5% of dry weight;
5. adding the aqueous phase to the final batch weight;
6. mixing to form a white opaque primary emulsion;
7. homogenizing the primary emulsion until no more visible oil droplets were visible using an optical microscope at 200× magnification;
8. passing each emulsion through a 0.2-micron filter;
9. filling the emulsions into glass vials and sealing the vials with rubber stoppers; and
10. placing each at −20, 2-8, 25 and 40° C.

The emulsions were tested for: appearance; pH; filterability by a 0.2-micron filter to determine if the emulsion contains aggregated glucagon or is too viscous to filter; and mean droplet size by dynamic light scattering using a Malvern Zetasizer Model Nano. The test results are summarized in TABLE 3.

TABLE 3
Physical Test Results and Total Droplet Surface Area

| | F-1 | F-2 | F-3 | F-4 |
|---|---|---|---|---|
| Appearance | Slightly | While | Slightly | Slightly |
| | yellow, | opaque, | yellow, | yellow, |
| | opaque, | uniform | uniform, | uniform, |
| | viscous | | translucent | translucent |
| pH | 6.48 | 6.93 | 6.78 | 7.46 |
| 0.2-micron | Yes, but | No, did not | Yes | No, lost |
| filterability | difficult due to | pass through | | glucagon |
| | high viscosity | the filter | | to the filter |
| Oily phase | 20.1 | 15.1 | 15.1 | 10.1 |
| concentration | | | | |
| (%) | | | | |
| Mean droplet | 154 | 210 | 118 | 136 |
| diameter (nm) | | | | |
| Total Droplet | 3.9E+06 | 2.1E+06 | 3.8E+06 | 2.2E+06 |
| Surface Area | | | | |
| (mm²) | | | | |

Only when the Total Droplet Surface Area (mm²) exceeded the Critical Droplet Surface Area (3.0 E+06 mm²), the nanoemulsion (F-1 or F-3) passed through the 0.2-micron filter and appeared to be free of glucagon aggregates. When the oily phase exceeded 20% by weight as in F-1, the emulsion became too viscous and difficult to filter so the oily phase concentration is preferred to be less 20% by weight. F-4 did not solubilize glucagon completely due to a low oily phase concentration (about 10%), which resulted in insufficient Total Droplet Surface Area (2.2 E+06 mm²) to solubilize 1 mg glucagon. F-3 (which contained about 1 mg/mL glucagon, 5% by weight oil, 10% by weight phospholipid with a mean droplet diameter of 118 nm and Total Droplet Surface Area of 3.8 E+06 mm² per milligram of glucagon solubilized) is a physically stable, filterable and glucagon aggregate-free nanoemulsion.

This study supports formation of a nanoemulsion containing egg lecithin, medium chain oil and an aqueous phase, wherein the total oily phase is between about 10 and 20% and oil concentration is no more than that of the phospholipid, and wherein the Total Droplet Surface Area exceeds the Critical Droplet Surface Area, is capable of solubilizing glucagon and forming a 0.2-micron-filterable liquid composition.

### Example 2

### HPLC Method and Glucagon Degradation Product Determination by HPLC

A reverse phase HPLC method was developed to test the concentration of glucagon and its degradation products in the nanoemulsion of the present invention. This method was used to evaluate the chemical stability of glucagon in a nanoemulsion. The HPLC method conditions were as follows. The HPLC gradient is summarized in Table 4.

Column: 4.6×250 mm, C-8

Mobile phase A: 0.05% by vol. trifluoroacetic acid in in water

Mobile phase B: 0.05% by vol. trifluoroacetic acid in in water in acetonitrile

Column temp: 35° C.

Wavelength: 214 nm

Autosampler temp: 5° C.

TABLE 4
HPLC Elution Gradient

| Time (min) | % Mobile Phase B |
|---|---|
| 0-16 | 28 |
| 20 | 30 |
| 21 | 60 |
| 22-30 | 100 |
| 31-40 | 28 |

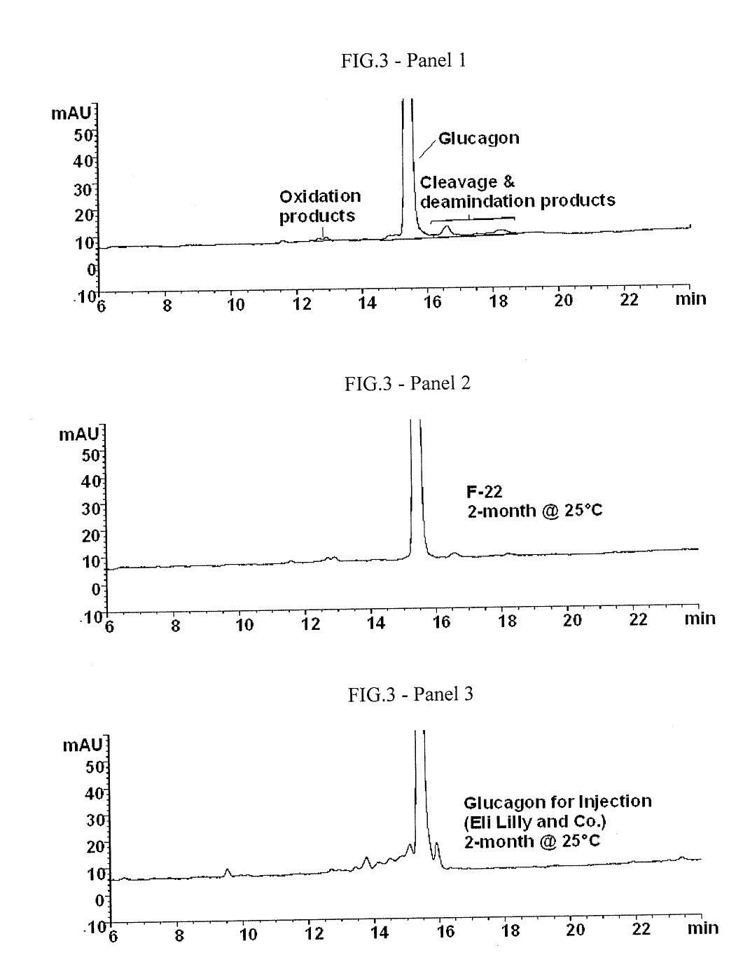

Representative HPLC chromatograms of a freshly prepared (Panel 1) and degraded glucagon (Panel 2) are shown in FIG. 3. This method is capable of separating and quantitating the major degradation products of glucagon, i.e., the numerous aspartic cleavage and glutaminyl deamidation degradation products and oxidation products (Panel 2). FIG. 3, Panel 3 shows the difference in amount of degradation products between a nanoemulsion of the present invention (F-22) with the Glucagon for Injection product from Eli Lilly and Company. As shown in this example, the developed HPLC analytical method reveals that glucagon is prone to form various degradation products in an aqueous environment and that the nanoemulsion (F-22) of the current invention exhibited superior chemical stability to that of the Glucagon for Injection product from Eli Lilly and Company.

### Example 3

### Improvement of Glucagon Chemical Stability in a Nanoemulsion at a Neutral pH

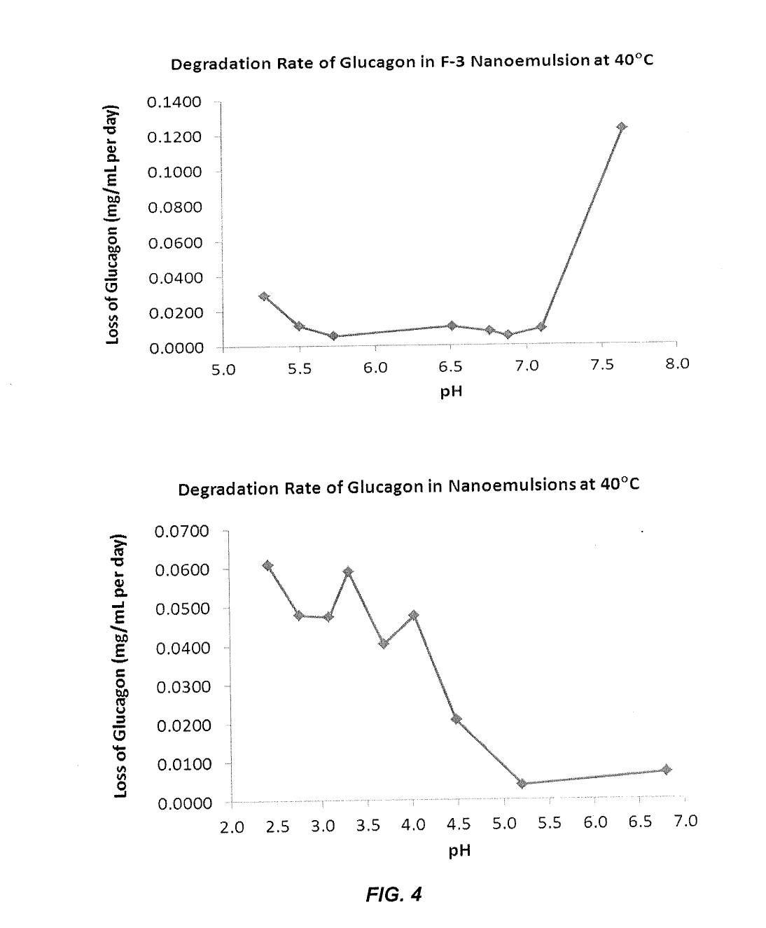

A new batch of the F-3 nanoemulsion composition of Example 1 was prepared and divided into several small portions. Each portion was adjusted with NaOH to a pH between pH 5 and pH 7.5, filled and sealed in a glass vial and placed at 40° C. to accelerate glucagon's chemical degradation. After 1, 11, 30 and 45 days, each composition was analyzed for glucagon concentration using the HPLC method as described in Example 2. An average rate of loss of glucagon was calculated and used to indicate the relative stability of glucagon over the pH range studied. TABLE 5 below shows the glucagon loss rate in mg/mL/day for the different pH values. The pH vs. loss rate profile is shown in FIG. 4 (upper panel).

TABLE 5
Glucagon concentration (mg/mL) in F-3
recovered after storage at 40° C.

| | Day | | | | | Avg. Glucagon Loss Rate |
|---|---|---|---|---|---|---|
| pH | 0 | 1 | 11 | 30 | 45 | (mg/mL per day) |
| 5.27 | 0.95 | 0.76 | 0.56 | | | 0.029 |
| 5.50 | 1.01 | 0.91 | 0.70 | 0.57 | 0.44 | 0.012 |
| 5.73 | 1.06 | 0.85 | 0.56 | 0.75 | 0.64 | 0.006 |
| 6.51 | 1.02 | 0.82 | 0.70 | 0.54 | 0.45 | 0.011 |
| 6.76 | 1.11 | 0.94 | 0.77 | 0.75 | 0.66 | 0.008 |
| 6.88 | 1.16 | 0.96 | 0.56 | 0.77 | 0.78 | 0.005 |
| 7.10 | 1.06 | 0.88 | 0.56 | 0.60 | 0.52 | 0.010 |
| 7.65 | 1.44 | 1.11 | 0.00 | | | 0.123 |

In a second pH vs. rate study, the F-3 nanoemulsion composition was prepared at a lower pH range (pH 2.4 to 6.8). The glucagon loss rate results are shown in TABLE 6 and the pH vs. loss rate profile is shown in FIG. 4 (lower panel).

TABLE 6
Glucagon loss rate

| Day | | | | |
|---|---|---|---|---|
| pH | 0 | 7 | 14 | Avg. Glucagon Loss Rate (mg/mL per day) |
| 2.42 | 1.40 | 1.26 | 0.55 | 0.061 |
| 2.76 | 1.36 | 1.25 | 0.69 | 0.048 |
| 3.09 | 1.36 | 0.81 | 0.70 | 0.047 |
| 3.31 | 1.41 | 1.15 | 0.59 | 0.059 |
| 3.69 | 1.36 | 1.24 | 0.80 | 0.040 |
| 4.03 | 1.42 | 0.94 | 0.75 | 0.048 |
| 4.48 | 1.42 | 1.42 | 1.13 | 0.021 |
| 5.20 | 1.36 | 1.35 | 1.30 | 0.004 |
| 6.80 | | | | 0.007 |

The data show that glucagon is more stable at a pH between pH 5.5 and pH 7.2. Below pH 5.5, which is the case for Eli Lilly and Co.'s Glucagon for Injection (pH 2-4), glucagon is less stable. Additionally, at a pH above 7.2, glucagon becomes much less stable. US Patent Application 2011/0097386 disclosed a lysophospholipid-solubilized glucagon composition at a pH that is as low as 4 or as high as 7.5 (Claim #1), which could negatively affect glucagon stability. For the nanoemulsion compositions of the present invention, the preferred pH range is from pH 2.4 to 7.2, or more preferably from 2.7 to 6.8.

### Example 4

### Improvement of Glucagon Chemical Stability by the Addition of Amino Acids to the Composition

The following compositions were prepared using the Example 1 method above except that no ethanol was used to dissolve the lecithin and oil.

TABLE 7
Composition (% wt)

| Component | F-3 | F-5 | F-6 |
|---|---|---|---|
| Glucagon | 0.1 | 0.1 | 0.1 |
| Egg lecithin | 10 | 10 | 10 |
| Medium chain oil | 5 | 5 | 5 |
| Glycine | | 0.15 | |
| Methionine | | | 0.15 |
| Aqueous phase* | 85 | 85 | 85 |
| Total | 100 | 100 | 100 |

*Aqueous phase = DI-water containing 10% wt sucrose and 0.0055% wt EDTA disodium dihydrate (pH not adjusted)

Each composition was filled and sealed in glass vials and placed at 40° C. to accelerate glucagon's chemical degradation. After 5 days, the compositions were analyzed for glucagon concentration using the HPLC method as described in Example 2 above. The glucagon recovered (% of the initial concentration) after 5 days in each composition is provided in the TABLE 8 below:

TABLE 8
Glucagon chemical stability

| | Glucagon |
|---|---|
| ID | Recovery (% of the initial concentration) |
| F-3 | 83.9 |
| F-5 (=F-3 + glycine) | 80.8 |
| F-6 (=F-3 + methionine) | 86.2 |

The data show that glucagon chemical stability can be either increased as by methionine or decreased as by glycine. This finding does not support the teaching by Kornfelt, et al. (U.S. Pat. No. 5,652,216), which claimed all amino acids (or ampholytes) can stabilize glucagon. Surprisingly, methionine stabilizes glucagon and it does it by a mechanism other than by being an ampholyte. As shown in the present Example, non-selective addition of an amino acid (such as glycine as taught by Kornfelt) to a glucagon emulsion could be undesirable because it can accelerate glucagon degradation.

### Example 5

### Improvement of Chemical Stability of a Glucagon Composition by Adding Antioxidants

The following compositions were prepared using the Example 1 method except that no ethanol was used to dissolve the lecithin and oil. The antioxidants were selected from reducing sugars and reducing amino acids.

TABLE 9
Composition (% wt)

| | | | F-9 | | | | |
|---|---|---|---|---|---|---|---|
| Component | F-7 | F-8 | (=F-3) | F-10 | F-11 | F-12 | F-13 |
| Glucagon | 0.10 | 0.10 | 0.10 | 0.10 | 0.10 | 0.10 | 0.10 |
| Egg lecithin | 10 | 10 | 10 | 10 | 10 | 10 | 10 |
| Medium chain | 5 | 5 | 5 | 5 | 5 | 5 | 5 |
| oil | | | | | | | |
| Methionine | 0.15 | 0.3 | 0.15 | 0.15 | 0.15 | 0.15 | 0.15 |
| Cysteine | | | | 0.1 | | | |
| Dextrose | | | | | 3.75 | | |
| (glucose) | | | | | | | |
| Fructose | | | | | | 5 | |
| Lactose | | | | | | | 4.9 |
| monohydrate | | | | | | | |
| Sucrose | 10 | 10 | 10 | 10 | | | |
| EDTA disodium | 0.0055 | 0.0055 | 0.0055 | 0.0055 | 0.0055 | 0.0055 | 0.0055 |
| dihydrate | | | | | | | |
| Total | 100 | 100 | 100 | 100 | 100 | 100 | 100 |

Each composition was filled and sealed in glass vials and placed at 40° C. to accelerate glucagon's chemical degradation. After 6 and 23 days, the compositions were analyzed for glucagon concentration using the HPLC method of Example 2. For each composition, an average rate of loss of glucagon was calculated and used to indicate the relative stability of glucagon in presence of an antioxidant. TABLE 10 lists the glucagon loss rate in mg/mL/day.

TABLE 10
Glucagon concentration (mg/mL) recovered after storage at 40° C.

| | Day | | | | Avg. |
|---|---|---|---|---|---|
| pH | ID | 0 | 6 | 23 | Glucagon Loss Rate (mg/mL per day) |
| 6.91 | F-7 | 1.00 | 0.86 | 0.76 | 0.009 |
| 6.80 | F-8 | 1.01 | 0.88 | 0.83 | 0.007 |
| 6.96 | F-9 | 1.41 | 1.18 | 0.97 | 0.018 |
| 6.94 | F-10 | 0.99 | 0.74 | 0.54 | 0.018 |
| 6.98 | F-11 | 1.02 | 0.83 | 0.65 | 0.015 |
| 6.87 | F-12 | 1.00 | 0.86 | 0.84 | 0.006 |
| 6.97 | F-13 | 1.10 | 0.82 | 0.00 | 0.046 |

The glucagon loss rate data suggested that certain selected antioxidants when added to a composition were able to slow down glucagon degradation while others accelerated it. Methionine, either alone or in combination with fructose or dextrose, appeared to be the most effective in stabilizing glucagon. Lactose, on the other hand, was detrimental to glucagon stability and therefore is undesirable.

### Example 6

### Improvement of Glucagon Chemical Stability by Reducing Non-Peptide, Water-Soluble Ion Content in a Nanoemulsion Composition

Water-soluble ions can be introduced into a nanoemulsion composition from at least three sources: (1) as counter-ions or residual ions from the glucagon raw material; (2) as counter-ions or residual ions from added inactive ingredients such as oil, phospholipids, antioxidants, etc.; and (3) the acid or base used to adjust the pH for the nanoemulsion. For example, the glucagon raw material used for this this study (BACHEM, lot 1017219) contains a total of 3.69% known ions (0.85% ammonium, 2.6% chloride and 0.24% acetate). The overall concentration of these “extraneous ions” in a nanoemulsion can be measured by the electrical conductivity of the nanoemulsion.

Since the extraneous ions are generally water soluble and will likely remain in the nanoemulsion's aqueous phase, it is therefore possible to remove them by exchanging the aqueous phase containing the extraneous ions with a low ionic content or ion-free aqueous solution using an ultrafiltration process. Ultrafiltration uses a membrane to retain the oil droplets while allowing the aqueous phase containing the extraneous ions to pass through. This study evaluates the overall concentration of such extraneous ions on the rate of degradation of glucagon in nanoemulsions. Three nanoemulsion compositions (F-22, F-23 and F-29, all at about pH 5.2) were prepared using a same method as in Example 4 and having the compositions shown below:

TABLE 11
Compositions (% wt) of F-22, F-23 and F-29

| Component | F-22 | F-23 | F-29 |
|---|---|---|---|
| Glucagon | 0.109 | 0.109 | 0.109 |
| Egg lecithin | 10 | 10 | 10 |
| Medium chain oil | 5 | 5 | 5 |
| Methionine | 0.3 | 0.3 | 0.3 |
| Sucrose | 10 | 10 | 10 |
| EDTA disodium dehydrate | 0.0055 | 0.0055 | 0.0055 |
| Di-water, added to | 100 | 100 | 100 |
| NaCl | | 0.058 | |
| Estimated total extraneous | 13.9* | 23.9** | ~0*** |
| ion concentration (mM) | | | |

*Contributed primarily by counter-ions and residual ions from the glucagon raw material, including ammonium, chloride and acetate to a total about 3.7% by weight of the raw material. F-22 was not adjusted for pH, therefore no acid or base was added.
**F-23 was same as F-22 except with 10 mM NaCl added, which was added to simulate the ions that would have been introduced into the nanoemulsion if the pH was adjusted using added acid and/or base (e.g., HCl and NaOH).
***The extraneous ions in F-29 were removed by an ultrafiltration process. The ultrafiltration was conducted using a centrifugal diafiltration filter (Ultracel by Amicon with a 3K MWCO membrane) for 3x volume exchange with an aqueous solution free of extraneous ions.

Each composition was filled and sealed in glass vials and placed at 37° C. to accelerate glucagon's chemical degradation. After 1, 3 and 7 days, the compositions were analyzed for glucagon concentration using the HPLC method of Example 2. Based on the loss of the glucagon, average rates of loss of glucagon were calculated and used to indicate the effect of the extraneous ion concentration on the stability of glucagon. For each composition, the overall ion concentration was measured using an electrical conductivity meter (Oakton, CON 11, Eutech Instruments) and expressed as that concentration of a NaCl solution having the same electrical conductivity as measured by the same meter. TABLE 12 below lists the glucagon loss rate in mg/mL/day and the measured electrical conductivity of each composition.

TABLE 12
Effect of ion concentration on glucagon degradation rate

| | | | Estimated |
|---|---|---|---|
| | | | Glucagon |
| | | Avg. | Concentration |
| | Electrical | Glucagon Loss | Loss after |
| | conductivity | Rate at 37° C. | 7 days at 37° C. |
| ID | (in NaCl conc. %) | (mg/mL per day) | (% of initial conc.) |
| F-22 | 0.055 | 0.009 | 6.3 |
| F-23 | 0.109 | 0.013 | 9.1 |
| F-29 | 0.001 | 0.004 | 2.8 |

This study showed, surprisingly, that extraneous ions promote, in a very significant way, glucagon chemical degradation. Compared to F-22, addition of a small amount of NaCl (0.058% or 10 mM) as in F-23, nearly doubled the measured electrical conductivity and resulted in about a 44% increase in the glucagon degradation rate. The removal of extraneous ions by ultrafiltration, on the other hand, reduced the electrical conductivity to an almost undetectable level and also reduced the rate of glucagon degradation by more than 50%. F-23, which has the measured electrical conductivity equivalent to a 0.11% NaCl solution, had about a 9% loss in its initial glucagon concentration after 7 days at 37° C. With a 10% decrease representing the maximum acceptable loss, the studies of this example show it is therefore necessary to minimize ion concentration in a glucagon nanoemulsion. This study therefore suggests that to prepare a chemically stable nanoemulsion for glucagon, one should (1) use a glucagon raw material having a reduced or ion-free content of counter-ions or residual ions, preferably, of less than 5% of the total weight of the raw material; (2) avoid introducing any un-needed salt or extraneous ions including any ionizable acid or base, such as HCl and NaOH, for adjusting pH; and/or (3) remove the extraneous ions from the composition by ultrafiltration. A preferred nanoemulsion for glucagon should have a measured electrical conductivity value of no more than that of a 0.12% NaCl solution, i.e., the Critical Ion Content Limit, at which limit the estimated loss of glucagon after 7 days at 37° C. is about 10%.

### Example 7

### Preparation of Liquid and Lyophilized Nanoemulsion Using a Microfluidizer

F-22 with the following composition was prepared at 40 g batch size having the following composition:

TABLE 13
F-22 composition

| | % wt |
|---|---|
| Glucagon raw material | 0.109* |
| Egg lecithin | 10 |
| Medium chain oil | 5 |
| Methionine | 0.3 |
| Sucrose | 10 |
| EDTA disodium dehydrate | 0.0055 |
| Deionized water | Add to 100%** |

*Equivalent to 1 mg/mL glucagon.
**Density of the composition is 0.997 g/mL at room temperature.

The formulation was prepared by:
1. weighing out edetate disodium dihydrate, methionine and sucrose into a clean glass bottle;
2. adding DI-water;
3. mixing to dissolve solids to obtain an aqueous phase;
4. weighing out glucagon in another container;
5. adding a calculated amount of the aqueous phase to achieve the final glucagon concentration and mixing well to disperse glucagon and form a primary emulsion;
6. passing the primary emulsion through a Microfluidizer (Model 110 EH, Microfluidics, Inc.) operating at up to 25K PSI pressure;
7. continuing the process until the mean droplet diameter as measured using a dynamic light scattering instrument (Nano Zetasizer, Malvern Instruments) is about 100 nm;
8. filtering the nanoemulsion through a 0.2-micron filter (Milliflip, Millipore Corporation);
9. filling the filtered nanoemulsion into glass vials, and crimp-sealing some vials with rubber stoppers for storage and stability analysis of F-22; and
10. lyophilizing some other vials containing the liquid F-22 using a shelf lyophilizer (Model Dura-dry/Dura-stop MP, FTS).

The electrical conductivity of the final F-22 nanoemulsion was determined to be equivalent to about 0.06% NaCl, which is below the Critical Ion Content Limit (i.e., an electrical conductivity equivalent to that for a 0.12% NaCl solution) and the Total Droplet Surface Area was calculated as 3.8 E+6 mm², which is above the Critical Droplet Surface Area (i.e., 3.0 E+6 mm² per milligram of glucagon). No pH adjustment with acid or base was performed during the manufacturing process.

The initial and stability test results of the liquid and lyophilized F-22 are summarized in the Tables 14-29.

TABLE 14
Stability of Liquid F-22 - Appearance

| Storage | Time | 1 | 2 | 3 | 6 | 12 |
|---|---|---|---|---|---|---|
| conditions | 0 | month | months | months | months | months |
| −20° C. | Slightly | Slightly less | Slightly less | Slightly | Slightly | White, more |
| | yellow, | transparent, | transparent, | yellow, less | yellow, less | opaque |
| | semi- | uniform, | uniform, | transparent, | transparent, | |
| | transparent | clean vial | clean vial | uniform, | uniform, | |
| | uniform | wall | wall | clean vial | clean vial | |
| | liquid, clean | | | wall | wall | |
| 2-8° C. | vial wall | No change | No change | No change | No change | White, more |
| | | | | | | opaque |
| 25° C. | | Slightly less | Slightly less | Slightly | Slightly | White, more |
| | | transparent, | transparent, | yellow, less | yellow, less | opaque |
| | | uniform, | uniform, | transparent, | transparent, | |
| | | clean vial | clean vial | uniform, | uniform, | |
| | | wall | wall | clean vial | clean vial | |
| | | | | wall | wall | |
| 30° C. | | Less | Less | Light | Light | Not tested |
| | | transparent, | transparent, | yellow, less | yellow, less | |
| | | uniform, | uniform, | transparent, | transparent, | |
| | | Clean vial | Clean vial | uniform, | uniform, | |
| | | wall | wall | Clean vial | Clean vial | |
| | | | | wall | wall | |
| 40° C. | | Less | Less | Light | Light | Not tested |
| | | transparent, | transparent, | yellow, less | yellow, less | |
| | | uniform, | uniform, | transparent, | transparent, | |
| | | Clean vial | Clean vial | uniform, | uniform, | |
| | | wall, | wall, | Clean vial | Clean vial | |
| | | Slightly | Slightly | wall | wall | |
| | | more | more | | | |
| | | viscous | viscous | | | |

TABLE 15
Stability of Liquid F-22 - Glucagon Assay
(mg/mL) by HPLC (as in Example 2)

| Storage | Time | 1 | 2 | 3 | 6 | 12 |
|---|---|---|---|---|---|---|
| conditions | 0 | month | months | months | months | months |
| −20° C. | 0.939 | 0.935 | 0.925 | 0.950 | 0.916 | 0.923 |
| 2-8° C. | | 0.941 | 0.933 | 0.951 | 0.931 | 0.926 |
| 25° C. | | 0.924 | 0.919 | 0.921 | 0.883 | 0.890 |
| 30° C. | | 0.911 | 0.893 | 0.897 | 0.870 | Not |
| | | | | | | tested |
| 40° C. | | 0.880 | 0.847 | 0.823 | 0.689 | Not |
| | | | | | | tested |

TABLE 16
Stability of Liquid F-22 - Assay Recovery (% over Time 0)

| Storage | Time | 1 | 2 | 3 | 6 | 12 |
|---|---|---|---|---|---|---|
| conditions | 0 | month | months | months | months | months |
| −20° C. | 100 | 99.6 | 98.4 | 101.1 | 97.5 | 98.3 |
| 2-8° C. | | 100.2 | 99.3 | 101.3 | 99.1 | 98.6 |
| 25° C. | | 98.4 | 97.8 | 98.0 | 94.0 | 94.8 |
| 30° C. | | 97.0 | 95.0 | 95.5 | 92.7 | Not |
| | | | | | | tested |
| 40° C. | | 93.7 | 90.2 | 87.6 | 73.3 | Not |
| | | | | | | tested |

TABLE 17
Stability of Liquid F-22 - Chromatographic Purity (% peak area)

| Storage | Time | 1 | 2 | 3 | 6 | 12 |
|---|---|---|---|---|---|---|
| conditions | 0 | month | months | months | months | months |
| −20° C. | 97.9 | 96.8 | 98.1 | 98.7 | 98.0 | 97.5 |
| 2-8° C. | | 97.3 | 98.1 | 98.8 | 98.0 | 97.5 |
| 25° C. | | 97.6 | 97.8 | 97.9 | 96.7 | 97.2 |
| 30° C. | | 97.6 | 96.9 | 96.9 | 96.2 | Not |
| | | | | | | tested |
| 40° C. | | 96.7 | 95.7 | 94.8 | 93.8 | Not |
| | | | | | | tested |

TABLE 18
Stability of Liquid F-22 - pH Determination

| Storage | Time | 1 | 2 | 3 | 6 | 12 |
|---|---|---|---|---|---|---|
| conditions | 0 | month | months | months | months | months |
| −20° C. | 5.6 | 5.5 | 5.6 | 6.2 | 6.5 | 6.1 |
| 2-8° C. | | 5.6 | 5.8 | 6.1 | 6.2 | 6.1 |
| 25° C. | | 5.5 | 5.4 | 5.6 | 5.4 | 6.2 |
| 30° C. | | 5.2 | 5.3 | 5.6 | 6.4 | Not |
| | | | | | | tested |
| 40° C. | | 5.1 | 5.1 | 4.8 | 4.4 | Not |
| | | | | | | tested |

TABLE 19
Stability of Liquid F-22 - Droplet Size (Mean Diameter,
nm) by Dynamic Light Scattering (sample diluted
20X with DI-water prior to measurement)

| Storage | Time | 1 | 2 | 3 | 6 | 12 |
|---|---|---|---|---|---|---|
| conditions | 0 | month | months | months | months | months |
| −20° C. | 118.9 | 159.9 | 164.4 | 174.1 | 185.5 | 254.7 |
| 2-8° C. | | 104.0 | 107.6 | 107.9 | 114.4 | 119.2 |
| 25° C. | | 123.3 | 131.9 | 138.4 | 140.5 | 144.9 |
| 30° C. | | 182.7 | 148.8 | 150.9 | 143.3 | Not |
| | | | | | | tested |
| 40° C. | | 183.2 | 197.9 | 200.6 | 417.7 | Not |
| | | | | | | tested |

TABLE 20
Stability of Liquid F-22 - Zeta Potential (mV) (sample
diluted 20X with DI-water prior to measurement)

| Storage | Time | 1 | 2 | 3 | 6 | 12 |
|---|---|---|---|---|---|---|
| conditions | 0 | month | months | months | months | months |
| −20° C. | −11.5 | −23.5 | −14.7 | −13.9 | −24.3 | −16.5 |
| 2-8° C. | | −13.7 | −12.6 | −12.3 | −22.8 | −15.2 |
| 25° C. | | −15.1 | −13.5 | −13.0 | −26.5 | −23.7 |
| 30° C. | | −15.9 | −14.9 | −14.5 | −28.5 | Not |
| | | | | | | tested |
| 40° C. | | −16.2 | −17.1 | −22.8 | −41.5 | Not |
| | | | | | | tested |

TABLE 21
Stability of Liquid F-22 - PFAT5 (vol % of
oversized globules larger than 5-microns*)

| Storage | Time | 1 | 2 | 3 | 6 | 12 |
|---|---|---|---|---|---|---|
| conditions | 0 | month | months | months | months | months |
| −20° C. | 0.014 | 0.030 | 0.029 | 0.004 | 0.003 | 0.006 |
| 2-8° C. | | 0.003 | 0.016 | 0.003 | 0.001 | 0.002 |
| 25° C. | | 0.003 | 0.010 | 0.003 | 0.003 | 0.002 |
| 30° C. | | 0.009 | 0.008 | 0.001 | 0.002 | Not |
| | | | | | | tested |
| 40° C. | | 0.006 | 0.006 | 0.002 | 0.011 | Not |
| | | | | | | tested |

*The United States Pharmacopeia has developed two methods and criteria (under Chapter <729>) to measure the mean droplet size (Method I), and the large-diameter tail >5 micron or PFAT5 (Method II) of the globule size distribution to verify the stability of lipid injectable emulsions. PFAT5 limit is being set at 0.05% by volume of the total oily phase and is considered to have greater implications for IV infusion safety. PFAT5 value is an indicator of presence of large particles including glucagon aggregates.

TABLE 22
Stability of Lyophilized F-22 - Appearance
(post-reconstitution with DI-water)

| Storage | Time | 1 | 2 | 3 | 6 | 12 |
|---|---|---|---|---|---|---|
| conditions | 0 | month | months | months | months | months |
| 25° C. | Slightly | No | No | No | No | No |
| | yellow, | change | change | change | change | change |
| | semi- | | | | | |
| | transparent, | | | | | |
| | uniform | | | | | |
| | liquid, clean | | | | | |
| | vial wall | | | | | |

TABLE 23
Stability of Lyophilized F-22 - Glucagon Assay (mg/vial)

| Storage | Time | 1 | 2 | 3 | 6 | 12 |
|---|---|---|---|---|---|---|
| conditions | 0 | month | months | months | months | months |
| 25° C. | 0.449 | 0.455 | 0.452 | 0.446 | 0.446 | 0.451 |

TABLE 24
Stability of Lyophilized F-22 - Assay Recovery (% over Time 0)

| Storage | Time | 1 | 2 | 3 | 6 | 12 |
|---|---|---|---|---|---|---|
| conditions | 0 | month | months | months | months | months |
| 25° C. | 100 | 101.3 | 100.7 | 99.2 | 99.4 | 100.5 |

TABLE 25
Stability of Lyophilized F-22 - Chromatographic
purity (% peak area)

| Storage | Time | 1 | 2 | 3 | 6 | 12 |
|---|---|---|---|---|---|---|
| conditions | 0 | month | months | months | months | months |
| 25° C. | 96.5 | 97.7 | 98.1 | 98.5 | 98.7 | 97.4 |

TABLE 26
Stability of Lyophilized F-22 - pH

| Storage | Time | 1 | 2 | 3 | 6 | 12 |
|---|---|---|---|---|---|---|
| conditions | 0 | month | months | months | months | months |
| 25° C. | 5.6 | 5.0 | 5.2 | 5.5 | 5.8 | 5.7 |

TABLE 27
Stability of Lyophilized F-22 - Droplet Size (mean
diameter, nm) by Dynamic Light Scattering (sample was
diluted 20X with DI-water prior to measurement)

| Storage | Time | 1 | 2 | 3 | 6 | 12 |
|---|---|---|---|---|---|---|
| conditions | 0 | month | months | months | months | months |
| 25° C. | 119.9 | 121.9 | 117.1 | 120.0 | 122.8 | 121.7 |

TABLE 28
Stability of Lyophilized F-22 - Zeta Potential (mV) (sample
was diluted 20X with DI-water prior to measurement)

| Storage | Time | 1 | 2 | 3 | 6 | 12 |
|---|---|---|---|---|---|---|
| conditions | 0 | month | months | months | months | months |
| 25° C. | −10.6 | −11.2 | −9.8 | −10.6 | −24.3 | −20.3 |

TABLE 29
Stability of Lyophilized F-22 - PFAT5 (%
of oversized globules above 5-microns)

| Storage | Time | 1 | 2 | 3 | 6 | 12 |
|---|---|---|---|---|---|---|
| conditions | 0 | month | months | months | months | months |
| 25° C./60% RH | 0.016 | 0.004 | 0.011 | 0.008 | 0.002 | 0.016 |

The above data indicate that F-22 meets all the critical requirements for a nanoemulsion according to this invention, i.e., having and maintaining (1) a neutral pH, (2) a mean droplet size less than 200 nm, (3) more than 50% glucagon associated with the oil droplets (Example 10), (4) a Total Droplet Surface Area greater than the Critical Droplet Surface Area, and (5) a total ion content below the Critical Ion Content Limit. Consequently, F-22 exhibited excellent physical and chemical stability. Physically, F-22, in the liquid and ready-to-inject form, retained its pH, semi-transparent appearance, submicron droplet size, droplet size distribution, and zeta potential at 2-8 or 25° C. without any sign of glucagon aggregation or precipitation. Chemically, F-22 showed no detectable loss of glucagon under the same storage conditions for 2 months. At 40° C., F-22 is stable for at least one month, suggesting sufficient stability for one week at body or near body temperature (30-37° C.) and suitability for use in an ambulatory bi-hormonal insulin/glucagon pump application. The lyophilized F-22 appears to be even more stable than the liquid F-22. The lyophilized F-22 may thus be considered as a room temperature product, capable of being reconstituted with water to become a ready-to-inject liquid and used for multiple times over many months.

### Example 8

### Demonstration of 7-Day Stability of Glucagon Nanoemulsion at Body Temperature

F-22 as described in Example 7 was tested for stability at body temperature (37° C.) for 7 days. The test results are shown in the table below:

TABLE 30
7-day, 37° C. Stability Evaluation of F-22 Liquid

| Day | 0 | 1 | 2 | 4 | 5 | 6 | 7 |
|---|---|---|---|---|---|---|---|
| Appearance | Slightly | No | No | Slightly | Slightly | Slightly | Slightly |
| | yellow, | change | change | more | more | more | more |
| | semi- | | | opaque | opaque | opaque | opaque |
| | transparent | | | | | | |
| Glucagon Assay by | 1.14 | 1.09 | 1.12 | 1.11 | 1.10 | 1.14 | 1.13 |
| HPLC (mg/mL) | | | | | | | |
| Assay Recovery (% | 100 | 95.7 | 98.0 | 96.8 | 96.1 | 100.1 | 99.1 |
| over Time 0) | | | | | | | |
| Glucagon Purity (% | 93.8 | 94.0 | 93.0 | 93.1 | 92.1 | 93.0 | 91.9 |
| peak area) | | | | | | | |
| Mean Droplet Dia. | | 128.9 | | | | | 166.7 |
| (nm) | | | | | | | |

The data from this study demonstrate a nanoemulsion prepared according to the present invention (e.g., F-22) is stable for 7 days at body temperature, making it suitable for use in a bi-hormonal insulin/glucagon pump application.

### Example 9

### Injectability Assessment of F-22 Nanoemulsion

The injectability of F-22 prepared according to the above Example 7 was evaluated. “Injectability” is a measurement of the peak force required needed to expel the liquid composition from a subcutaneous needle/syringe set at a fixed rate. The force was measured by a digital force gauge (Model HP-50, Beijing Lanetech Instruments Co., Ltd). The subcutaneous needle/syringe configuration consisted of a ½ mL BD Lo-Dose™ U-100 insulin syringe with 28 G×½ in BD Micro-Fine™ IV (Orange) permanently attached needle. The injection rate was set at about 0.9 mL/mL using a syringe pump. The peak force results are listed in the Table below:

TABLE 31
Injectability assessment of F-22 nanoemulsion

| Peak Injection | | | | |
|---|---|---|---|---|
| Force (lb) | Reading #1 | Reading #2 | Reading #3 | Avg. |
| Initial | 0.993 | 1.066 | 0.954 | 1.004 |
| After storage at | 0.980 | 1.050 | 0.974 | 1.001 |
| 37° C. for 9 days | | | | |

F-22 requires a very modest peak force to inject the nanoemulsion from an insulin syringe through a very fine subcutaneous needle. The peak injection force of about one pound can be easily self-applied manually by the user or applied by a medical device pump. The injectability (as measured by peak force) did not change after storage for 9 days at 37° C., suggesting the absence of any viscosity change in the formulation after exposure to these conditions.

### Example 10

### Partitioning of Glucagon Between the Oil and Aqueous Phases of the F-22 Nanoemulsion

This study was conducted to determine the concentration of glucagon in the aqueous phase of the F-22 nanoemulsion in order to calculate the ratio or partitioning of glucagon between the aqueous and oily phases or the percentage of glucagon remaining in the aqueous phase. The aqueous phase of the F-22 nanoemulsion as prepared in Example 7 was separated from the oily phase using a filter with a 3,000 MWCO membrane (Amicon Ultra 0.5 mL centrifugal filter with an Ultracel 3,000 MWCO membrane, Millipore Corp). The separation was achieved by centrifugation. Centrifugal filtration of F-22 removed about 50% of its weight. The collected filtrate, which was clear and colorless, was tested for glucagon content using the analytical method of Example 2.

TABLE 32
Glucagon Content in the Aqueous Phase of F-22

| | Glucagon | Percent Glucagon in |
|---|---|---|
| | concentration | the Aqueous Phase |
| Sample ID | (mg/mL) | (% over total) |
| Aqueous phase | 0.01 | 1% |
| F-22 | 1.04 | 100% |

The results show that only a residual level (about 1%) glucagon was found in the aqueous phase of F-22 and >99% of the glucagon is associated with or in the oily phase. This finding clearly indicates that, in the nanoemulsion composition of the present invention, glucagon is essentially absent from the aqueous phase. This observation is novel and contrasts all prior art glucagon compositions in which glucagon is solubilized in aqueous solutions (e.g., Great Britain Patent 1202607; Schneider A. B. and Edelhoch, H. J. 1972. Biol. Chem. 247: 4986-4991; Robinson, R. M., et al. 1982. Biopolymers 21: 1217-1228; Wu, C.-S. C. and Yang, J. T. 1978. Biochemistry 19: 2117-2122; Brown, L. R. and Wuthrich, K. 1980. Biochim. Biophys. Acta 603: 298-312; Matilainen, L., et al. 2008. J. Pharm Sci. 97(7):2720-9; Matilainen, L. et al. 2009. Eur. J. Pharm Sci. 36(4-5):412-20; European Patent 1061947; US Patent Application 2011/0097386; U.S. Pat. No. 5,652,216; among others).

### Example 11

### Composition, Preparation and Stability of F-28 (F-22 with Added m-Cresol)

A nanoemulsion composition containing an antimicrobial preservative (F-28) was prepared by adding 0.25% by weight m-cresol to the F-22 composition that was prepared using the method in Example 7. F-22 and F-28 were tested side-by-side for their stability after storage at 37° C. and the data are summarized in Tables 33-36.

TABLE 33
Stability of F-28 (F-22 + m-cresol) - Appearance

| ID | Time 0 | 1 day | 3 days | 7 days |
|---|---|---|---|---|
| F-22 | Light yellow, | No change | No change | No change |
| | semi-transparent | | | |
| F-28 | Same as F-22 | No change | No change | No change |

TABLE 34
Stability of F-28 (F-22 + m-cresol) -
Glucagon Assay (mg/mL) by HPLC

| ID | Time 0 | 1 day | 3 days | 7 days |
|---|---|---|---|---|
| F-22 | 1.04 | 0.99 | 1.00 | 0.97 |
| F-28 | 1.03 | 1.00 | 0.99 | 0.96 |

TABLE 35
Stability of F-28 (F-22 + m-cresol) -
Glucagon Assay Recovery (% over Time 0)

| ID | Time 0 | 1 day | 3 days | 7 days |
|---|---|---|---|---|
| F-22 | 100.0 | 95.6 | 96.1 | 93.6 |
| F-28 | 100.0 | 96.8 | 95.7 | 93.2 |

TABLE 36
Stability of F-28 (F-22 + m-cresol) -
Chromatographic purity (% peak area)

| ID | Time 0 | 1 day | 3 days | 7 days |
|---|---|---|---|---|
| F-22 | 98.41 | 96.7 | 96.0 | 95.9 |
| F-28 | 98.99 | 98.9 | 98.7 | 98.1 |

These results show that F-28 has a stability profile at 37° C. that is comparable to F-22, indicating that an antimicrobial preservative such as m-cresol can be added to the nanoemulsion of the present invention without affecting the stability of glucagon in the nanoemulsion.

### Example 12

### Preparation and Stability Comparison of F-22 and Glucagon for Injection (rDNA Origin) from Eli Lilly and Co.

The stability of F-22 was tested side-by-side with a commercial glucagon preparation (Glucagon for Injection (rDNA origin), Eli Lilly and Co., lot LDJF01EA) (the “Lilly Glucagon”) employing the same HPLC method as described in Example 2. The Lilly Glucagon is provided in a 2-part kit. One vial contains the glucagon lyophile and an accompanying syringe contains the acid solution diluent for reconstitution. The stability study of this example was conducted using the lyophilized form of the Lilly Glucagon and compared with F-22 at the same storage temperature (25° C.). The HPLC results are shown in TABLEs 37 and 38 and representative HPLC chromatograms are provided in FIG. 3.

TABLE 37
Stability Comparison of F-22 and the Lilly Glucagon -
Assay recovery (% over Time 0)

| ID | Time 0 | 1 month | 2 months | 3 months |
|---|---|---|---|---|
| Lilly Glucagon (lyophilized) | 100 | 87.1 | 89.7 | 81.3 |
| F-22, liquid | 100 | 98.4 | 97.8 | 98.0 |
| F-22, lyophilized | 100 | 101.3 | 100.7 | 99.2 |

TABLE 38
Stability Comparison of F-22 and the Lilly Glucagon -
Chromatographic purity (% peak area)

| ID | Time 0 | 1 month | 2 months | 3 months |
|---|---|---|---|---|
| Lilly Glucagon (lyophilized) | 92.0 | 91.4 | 93.7 | 93.4 |
| F-22, liquid | 97.9 | 97.6 | 97.8 | 97.9 |
| F-22, lyophilized | 96.5 | 97.7 | 98.1 | 98.5 |

This stability comparison clearly demonstrated that the glucagon nanoemulsion of the present invention (F-22), in either the liquid or lyophilized form, was chemically more stable than the commercial drug Glucagon for Injection (rDNA origin, Eli Lilly and Co.). The Lilly Glucagon has a very limited physical stability; with the glucagon aggregating within just a few hours after the lyophile was reconstituted using the provided acidic (pH 2-4) solution.

### Example 13

### Stability Comparison of F-22 and BIOD901 Formulation

The stability of F-22 compared to that of a lysophospholipid-solubilized, pH neutral solution formulation, as taught in US Patent Application 2011/0097386. This formulation (BIOD901) was prepared according to Example 3 in the aforementioned patent application. BIOD901 contains 1 mg/mL glucagon, 2 mg/mL lyso-myristoyl-phosphocholine (LMPC), 45 mg/mL glucose, 2 mg/mL m-cresol and is made in a basic solution, which is subsequently adjusted to pH 7. An alkali (e.g., NaOH) was used to adjust the pH upwards (>7) first and then an acid (e.g., HCl) was used to bring the pH back down to 7. BIOD901 was a clear solution initially but turned hazy with precipitates after 1 day of storage at 37° C. For the comparison, F-22 and BIOD901 were stored side-by-side at 37° C. for 7 days and analyzed using the same HPLC method as described in Example 2. The HPLC results are shown in TABLE 39.

TABLE 39
F-22 and BIOD901 Stability Comparison -
Assay Recovery (% over Time 0) After
Storage at 37° C.

| ID | Time 0 | 1 day | 3 days | 7 days |
|---|---|---|---|---|
| F-22 | 100.0 | 95.6 | 96.1 | 93.6 |
| BIOD901 #1 | 100.0 | 60.5* | 78.5* | 87.1* |

*Precipitation and loss of uniformity observed.

EXAMPLE 13 demonstrated that for the nanoemulsion composition of this invention, glucagon stability was substantially better than in a prior art solution composition that utilized water-soluble lysophospholipids to solubilize glucagon (US Patent Application 2011/0097386). Thus, F-22 will be better able to provide sufficiently stability at body or near body temperature (30-37° C.) to enable its use in a multiple-day pump delivery format without the use of potentially irritating lysophospholipids.

### Example 14

### Prophetic Compositions, Preparation and Uses Thereof

The following compositions have been contemplated to provide chemically and physically stable and pH neutral nanoemulsions for glucagon. Each composition can be prepared using the same or similar process as described in Example 1 or 7 to produce a Total Droplet Surface Area greater than the Critical Droplet Surface Area and overall ion content less than the Critical Ion Content Limit. Optionally these compositions can be lyophilized for further improved stability. Each composition can be used for hypoglycemia rescue, multiple dosing or in a therapeutic pump application.

TABLE 40
Glucagon-containing nanoemulsion compositions comprising other phospholipids, oils or sugars (% w/v)

| Component | F-30 | F-31 | F-32 | F-33 | F-34 | F-35 | F-36 | F-37 | F-38 | F-39 | F-40 | F-41 | F-42 |
|---|---|---|---|---|---|---|---|---|---|---|---|---|---|
| Glucagon | 0.1 | 0.1 | 0.1 | 0.1 | 0.1 | 0.1 | 0.1 | 0.1 | 0.1 | 0.1 | 0.1 | 0.1 | 0.1 |
| A synthetic | 10 | 10 | 10 | 10 | 10 | 10 | 10 | | | | | | |
| phospholipid (DMPC) | | | | | | | | | | | | | |
| A synthetic | | | | | | | | 10 | | | | | |
| phospholipid (POPC) | | | | | | | | | | | | | |
| A synthetic | | | | | | | | | 10 | | | | |
| phospholipid (DSPC) | | | | | | | | | | | | | |
| Soy lecithin | | | | | | | | | | 10 | | | |
| Egg lecithin | | | | | | | | | | | 10 | 10 | 10 |
| A vegetable oil | 5 | | | | | | | | | | | | |
| (sesame oil) | | | | | | | | | | | | | |
| A vegetable oil | | 5 | | | | | | | | | | | |
| (soybean oil) | | | | | | | | | | | | | |
| A vegetable oil | | | 5 | | | | | | | | | | |
| (corn oil) | | | | | | | | | | | | | |
| A synthetic oil | | | | 5 | | | | | | | | | |
| (tricaprylin) | | | | | | | | | | | | | |
| A synthetic oil | | | | | 5 | | | | | | | | |
| (triolein) | | | | | | | | | | | | | |
| A synthetic oil | | | | | | 5 | | | | | | | |
| (trimyristin) | | | | | | | | | | | | | |
| Medium chain oil | | | | | | | 5 | 5 | 5 | 5 | 1.25 | 1 | 0.5 |
| Methionine | 0.3 | 0.3 | 0.3 | 0.3 | 0.3 | 0.3 | 0.3 | 0.3 | | | 0.6 | 1 | 1 |
| Glycerol | | | | | | | | | | | | | 2.25 |
| Sucrose | 9 | 9 | 9 | 9 | 9 | 9 | 9 | 9 | 9 | 9 | 10 | 10 | |
| EDTA disodium | 0.0055 | 0.0055 | 0.0055 | 0.0055 | 0.0055 | 0.0055 | 0.0055 | 0.0055 | 0.0055 | 0.0055 | 0.0055 | 0.0055 | 0.0055 |
| dihydrate | | | | | | | | | | | | | |
| m-cresol | 0.25 | 0.25 | 0.25 | 0.25 | 0.25 | 0.25 | 0.25 | 0.25 | 0.25 | 0.25 | 0.25 | 0.25 | 0.25 |

| | |
|---|---|
| Water for injection | Add to 100% |

TABLE 41
Nanoemulsion compositions having different concentrations of glucagon (% w/v)

| Component | F-43 | F-44 | F-45 | F-46 | F-47 | F-48 | F-49 | F-50 | F-51 | F-52 | F-53 | F-54 | F-55 |
|---|---|---|---|---|---|---|---|---|---|---|---|---|---|
| Glucagon | 0.1 | 0.1 | 0.1 | 0.1 | 0.1 | 0.1 | 0.02 | 0.04 | 0.06 | 0.08 | 0.1 | 0.12 | 0.14 |
| Egg lecithin | 8 | 8 | 12 | 14 | 16 | 18 | 10 | 10 | 10 | 10 | 10 | 10 | 10 |
| Medium chain oil | 8 | 5 | 2.5 | 1.25 | 0.5 | 2 | 5 | 5 | 5 | 5 | 5 | 5 | 5 |
| Methionine | 0.3 | 0.3 | 0.3 | 0.3 | 0.3 | 0.3 | 0.3 | 0.3 | | | | | |
| Sucrose | 9 | 9 | 9 | 9 | 9 | 9 | 9 | 9 | 9 | 9 | 9 | 9 | |
| EDTA disodium | 0.0055 | 0.0055 | 0.0055 | 0.0055 | 0.0055 | 0.0055 | 0.0055 | 0.0055 | 0.0055 | 0.0055 | 0.0055 | 0.0055 | 0.0055 |
| dihydrate | | | | | | | | | | | | | |
| m-cresol | 0.25 | 0.25 | 0.25 | 0.25 | 0.25 | 0.25 | 0.25 | 0.25 | 0.25 | 0.25 | 0.25 | 0.25 | 0.25 |

| | |
|---|---|
| Water for injection | Add to 100% |

### Example 15

### Pharmacodynamic Study in Mice of F-22 and Glucagon for Injection (rDNA Origin, Eli Lilly & Company)

The objective of this study was to compare pharmacodynamics or PD (i.e., blood glucose versus time) profiles between F-22 and Glucagon for Injection (rDNA origin, Eli Lilly & Company lot A836687C) following subcutaneous injection in mice. A total of 8 C57BL/6, male, 8-9 week-old mice with body weights of 20-23 g were used in this study. The animals were acclimated and tail bleeds daily for 5 days prior to drug dosing. Mice were randomized into 2 groups and fasted for 16 hours. Food was be removed but water was available throughout the study. Blood glucose was measured on tail bleed samples using a handheld glucose meter (OneTouch Ultra, Lifescan, Milpitas, Calif.). Blood samples were taken following snipping off a small section of the tail tip (1-2 mm) with a pair of scissors. A blood sample (5-10 μL) was collected directly onto a glucose test strip. Generally, the first drop of blood was discarded and the second drop was tested. F-22 or the Lilly Glucagon was diluted to 20 μg/mL in normal saline and administered at 200 μg/kg dose within 1 hour after dilution. A baseline glucose sample (pre-dose) was taken 5 min prior to glucagon injection. Blood glucose concentration was measured at 10 min, 20 min, 30 min, 45 min, 60 min, 90 min, 120 min, 180 and 240 min post injection. All animals were examined for injection site reactions and no sign of any inflammation or infection was observed within 5 days after injection.

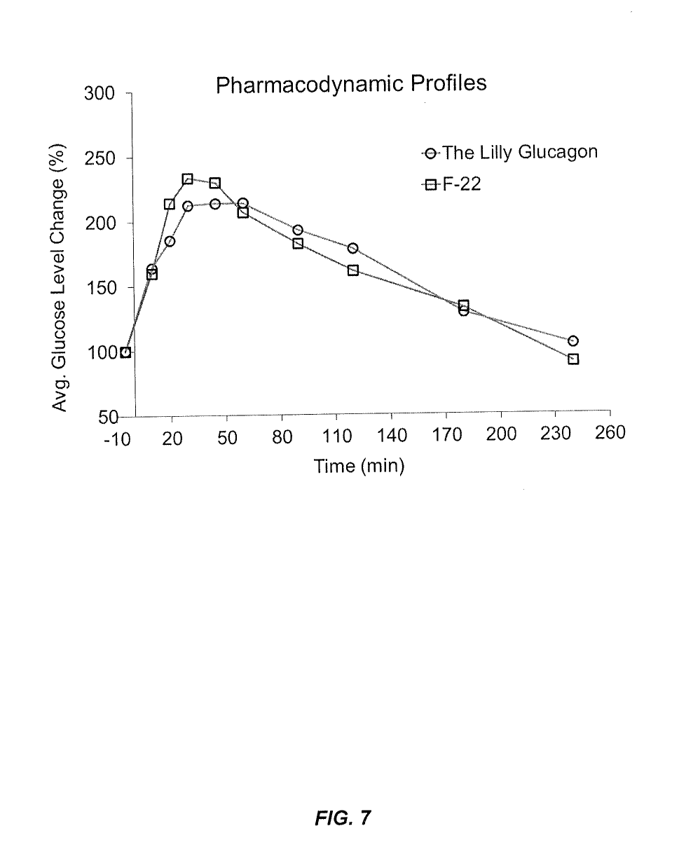

This study demonstrated comparable PD profiles for the two test formulations (FIG. 7), suggesting that the nanoemulsion composition (F-22) and the commercial drug (Glucagon for Injection (rDNA origin, Eli Lilly and Co.) demonstrate the same pharmacodynamics or pharmacokinetic properties.

### Example 16

### Admixture

A Nanoemulsion Vehicle for Use in Preparation of an Admixture Comprises the Following Inactive Ingredients:

TABLE 42
Composition of Nanoemulsion Vehicle for Admixture

| Ingredient | % w/v |
|---|---|
| Phospholipid (synthetic or lecithin) | 1 to 10 |
| Oil (medium chain oil, a vegetable oil, | 1 to 10 |
| or a combination thereof) | |
| Methionine | 0-0.5 |
| EDTA (or EDTA salt, e.g. sodium) | 0-0.1 |
| Water | Add to 100 |

The manufacturing process to make the nanoemulsion vehicle comprise the following steps:
1. Dissolve EDTA and methionine in water to form an aqueous phase.
2. Combine the oil and phospholipid to form an oil phase.
3. Homogenize to form a primary oil-in-water emulsion.
4. Pass the primary emulsion through a Microfluidizer until the mean oil droplet diameter is less than 200 nm.
5. Adjust the pH to 2 to 7 with HCl and/or NaOH.
6. Filter the nanoemulsion through a 0.2-micron filter to sterilize the nanoemulsion vehicle.
The nanoemulsion vehicle prepared this way is safe for injection and stable for 1-4 years at 2-8 or 25° C.

A dry glucagon formulation for use in preparation of the admixture may comprise the following ingredients:

TABLE 43
Composition of Dry Glucagon Formulation for Admixture

| Ingredient | Mg/vial |
|---|---|
| Glucagon | 0.1 to 30 |
| Lactose | 0 to 300 |
| HCl | As needed |

The manufacturing process to make the dry glucagon formulation may comprise the following step:
1. Weigh out dry glucagon drug substance into a vial and seal the vial.
Or
2. Weigh out dry glucagon drug substance and other dry pharmaceutically acceptable inactive ingredients into a vial and seal the vial.
Or
3. Dissolve glucagon, lactose and/or other pharmaceutically acceptable inactive ingredients in water
4. Adjust the pH to 1.5 to 5
5. Filter through a 0.2-micron filter to sterilize
6. Fill into vials (0.1 to 30 mg glucagon per vial)
7. Lyophilize to form a dry formulation

The dry glucagon formulation prepared this way is safe for injection and stable for 1-4 years at 2-8 or 25° C. The glucagon lyophile provided in the glucagon rescue kits marketed by Novo Nordisk or Eli Lilly may be used as the dry glucagon formulation.

An admixture comprises the following ingredients:

TABLE 44
Composition of Admixture

| Ingredient | % (w/v) | Mg/mL |
|---|---|---|
| Glucagon | 0.01-1 | 0.1-10 |
| Phospholipid (synthetic or lecithin) | 1 to 17.5 | 10-175 |
| Oil (medium chain oil, a vegetable oil, | 0.5 to 5 | 5-50 |
| or a combination thereof) | | |
| Methionine | 0-0.5 | 0-5 |
| EDTA (or a salt) | 0-0.1 | 0-1 |
| Water | QS* | QS* |

*Add sufficient quantity to the final volume

The preparation procedure to make the admixture comprises the following steps:
1. Combine a pre-determined amount of nanoemulsion vehicle (e.g. 1 mL) with a pre-determined amount of the dry glucagon formulation (e.g. 1 mg glucagon)
2. Mix for a few seconds (e.g. less than 1 minute) to form a uniform and translucent admixture in the final composition.

The admixture prepared this way is safe for injection and stable for 1-6 months at 2-8 or 1-4 weeks at 37° C. The mixing ratio of the nanoemulsion vehicle with the dry glucagon formulation may be adjusted to obtain a desired final glucagon concentration or dose.

In certain aspects, the present invention provides a process for preparing a nanoemulsion, the method comprising:

(a) preparing a nanoemulsion vehicle;
(b) preparing a dry glucagon formulation; and
(c) mixing the nanoemulsion vehicle and the dry glucagon formulation wherein the glucagon is not aggregated and retains no less than 75% of its concentration after 3-7 days storage at 37° C., and wherein the oily phase is in the form of oil droplets having a mean diameter of less than about 200 nm.

In certain aspects, the oily phase is between about 1% and 20% by weight of the nanoemulsion and the phospholipid concentration is no less than the oil concentration.

In certain aspects, the nanoemulsion has a the pH is between about 2 to about 7.5.

In certain aspects, the nanoemulsion comprises between about 0.25 and 1.5 mg/mL glucagon, between about 1 and 17.5% by weight phospholipid, between about 0.5 and 5% by weight oil, and an aqueous phase, wherein said nanoemulsion the oil droplets have a mean diameter of less than about 200 nm and, wherein, the glucagon is not aggregated and retains no less than 75% of its concentration after 3-7 days storage at 37° C.

The admixture is administered to a patient using a syringe/needle or auto-injector by injection or using a subcutaneous infusion pump (such an insulin pump) through a catheter. Preliminary PD/PK study has demonstrated activity of an admixture administered by an insulin pump in reversing hypoglycemic events induced by insulin in type-1 diabetic swine.

Modifications and variations of the present invention will be obvious to those skilled in the art from the foregoing detailed description and are intended to fall within the scope of the following claims. The teachings of all references cited herein are specifically incorporated by reference.

What is claimed is:

1. An oil-in-water nanoemulsion composition comprising:
glucagon,
an oily phase, and
an aqueous phase,
wherein the glucagon is not aggregated and retains no less than 75% of its concentration after 3-7 days storage at 37° C., and wherein the oily phase is in the form of oil droplets having a mean diameter of less than about 200 nm.

2. The nanoemulsion according to claim 1, wherein the oily phase comprises a phospholipid and an oil.

3. The nanoemulsion according to claim 2, wherein the oily phase is between about 10% and 20% by weight of the nanoemulsion and the phospholipid concentration is no less than the oil concentration.

4. The nanoemulsion according to claim 1, wherein no less than 50% of the glucagon is non-covalently associated with the oily phase.

5. The nanoemulsion according to claim 1, wherein said nanoemulsion is made with egg or soy lecithin having a lysolecithin content of less than 5% of the lecithin weight.

6. The nanoemulsion according to claim 2, wherein the oil is a medium chain oil, a vegetable oil, or a combination thereof.

7. The nanoemulsion according to claim 1, wherein the pH of said nanoemulsion is between about 2.7 to about 7.5.

8. The nanoemulsion according to claim 1, wherein the nanoemulsion is filterable through a 0.2-micron filter.

9. The nanoemulsion according to claim 1, wherein said nanoemulsion further contains at least one antioxidant selected from the group consisting of EDTA, methionine, lactose, fructose, dextrose, cysteine, glutathione or salt or a combination thereof.

10. The nanoemulsion according to claim 1, further comprising an antimicrobial preservative selected from the group consisting of EDTA, benzyl alcohol, a paraben, sodium metabisulfite, a cresol, or a salt or a combination thereof.

11. The nanoemulsion according to claim 1, wherein the composition is provided in a vial, a syringe or auto-injector.

12. The nanoemulsion according to claim 1, wherein the glucagon is administered from a pump or from a syringe via a needle through a subcutaneous, intramuscular or intravenous route.

13. The nanoemulsion according to claim 1, wherein the nanoemulsion is ready-to-inject.

14. A nanoemulsion comprising between about 0.25 and 1.5 mg/mL glucagon, a phospholipid, between about 0.5 and 5% by weight oil, and an aqueous phase, wherein said nanoemulsion the oil droplets have a mean diameter of less than about 200 nm and wherein the glucagon is not aggregated and retains no less than 75% of its concentration after 3-7 days storage at 37° C.

15. A nanoemulsion comprising about 1 mg/mL glucagon, about 10% by weight lecithin, about 0.5 to 5% by weight medium chain oil, and an aqueous phase, wherein said nanoemulsion the glucagon is not aggregated and retains no less than 75% of its concentration after 3-7 days storage at 37° C., and wherein the pH is between about 2.7 to about 7.5.

16. A process for preparing a nanoemulsion, the method comprising:
(a) combining glucagon and an aqueous phase;
(b) adding phospholipid and oil;
(c) mixing and homogenizing to form a nanoemulsion having an average droplet size of no more than 200 nm in diameter; and
(d) passing said nanoemulsion through a 0.2-micron filter.
